SEQUENCE LISTING (13 sequences)

SEQ ID NO: 1 - DNA, 1497 bp, Coleus forskohlii
atggagttggtggaagtgattgtggtggtggtgggagcggccgcgttgggcgttgttttgtggtctcacttgaagccggagggcagaaagcttccaccagggccttcccctctgcctatcttcggaaacattttccagctgacgggtccaaacacttgtgaatcatttgcgaacctctccaagaaatacggccccgtcatgtccttacgcctcggtagcttattcaccgtcgtcatttcgtcgccggaaatggcgaaagaagtactgactaacacagactttttagagaggcccctgatgcaggcggtccatgcgcatgaccacgcccagttctccattgcgttcctgccggtgaccacccctaaatggaaacaactgcgccggatttgccaggagcaaatgtttgcgagtcggatcttggagaagagccagcccctccgtcaccagaagctgcaggagctgatcgaccacgtgcagaaatgctgcgacgctgggcgagcggttactatccgcgacgccgccttcgcgaccacgctcaacctcatgtcggtgacgatgttctcggctgacgctactgagttggattccagcgtcaccgcggagttgagggagctcatggcgggcgtcgttactgttcttggcactccgaatttcgccgacttcttccccatcctcaaatatttggatccgcaaggggtgaggcgcaaggcacatttccactatgggaagatgtttgaccacatcaagagtcggatggccgagagagtcgagttgaaaaaggcaaacccaaatcatctcaaacacgacgacttcttggagaaaatcttggacatcagtctacggagggactacgagttgaccatccaggacattacgcatttactggtggacttgtatgtagcaggaagcgaatcaacggtaatgtcaattgaatggataatgtctgaattgatgctgcacccgcagtcactagcgaagctgaaagccgagctgagaagcgtgatgggagagagaaagatgatccaggaatcagaggacatatcaaggcttccctttctgaatgccgtcatcaaagaaaccctccgtctccacccgccaggtcccctcctgtttccccgccaaaacaccaacgatgtggaactcaatggctatttcatcccaaaaggtactcagatacttgttaacgaatgggcaataggcagagatcccagtgtttggcccaatccagaatcttttgtgccggaacgcttcttggacaagaacattgattacaaaggccaagatccccagctcgtcccatttggatcgggtagaaggatctgtcttgggatacccatagcgcatcggatggtgcattctacagtggccgcattaattcacaatttcgagtggaagttcgccccagacggcagtgaatataacagggagttgtttagcgggccagcgctgcgcagggaagttcctctcaacctcatcccactcaacccttcattttga

SEQ ID NO: 2 - protein, 498 aa, Coleus forskohlii
MELVEVIVVVVGAAALGVVLWSHLKPEGRKLPPGPSPLPIFGNIFQLTGPNTCESFANLSKKYGPVMSLRLGSLFTVVISSPEMAKEVLTNTDFLERPLMQAVHAHDHAQFSIAFLPVTTPKWKQLRRICQEQMFASRILEKSQPLRHQKLQELIDHVQKCCDAGRAVTIRDAAFATTLNLMSVTMFSADATELDSSVTAELRELMAGVVTVLGTPNFADFFPILKYLDPQGVRRKAHFHYGKMFDHIKSRMAERVELKKANPNHLKHDDFLEKILDISLRRDYELTIQDITHLLVDLYVAGSESTVMSIEWIMSELMLHPQSLAKLKAELRSVMGERKMIQESEDISRLPFLNAVIKETLRLHPPGPLLFPRQNTNDVELNGYFIPKGTQILVNEWAIGRDPSVWPNPESFVPERFLDKNIDYKGQDPQLVPFGSGRRICLGIPIAHRMVHSTVAALIHNFEWKFAPDGSEYNRELFSGPALRREVPLNLIPLNPSF

SEQ ID NO: 3 - protein, 773 aa, Coleus forskohlii
MKMLMIKSQFRVHSIVSAWANNSNKRQSLGHQIRRKQRSQVTECRVASLDALNGIQKVGPATIGTPEEENKKIEDSIEYVKELLKTMGDGRISVSPYDTAIVALIKDLEGGDGPEFPSCLEWIAQNQLADGSWGDHFFCIYDRVVNTAACVVALKSWNVHADKIEKGAVYLKENVHKLKDGKIEHMPAGFEFVVPATLERAKALGIKGLPYDDPFIREIYSAKQTRLTKIPKGMIYESPTSLLYSLDGLEGLEWDKILKLQSADGSFITSVSSTAFVFMHTNDLKCHAFIKNALTNCNGGVPHTYPVDIFARLWAVDRLQRLGISRFFEPEIKYLMDHINNVWREKGVFSSRHSQFADIDDTSMGIRLLKMHGYNVNPNALEHFKQKDGKFTCYADQHIESPSPMYNLYRAAQLRFPGEEILQQALQFAYNFLHENLASNHFQEKWVISDHLIDEVRIGLKMPWYATLPRVEASYYLQHYGGSSDVWIGKTLYRMPEISNDTYKILAQLDFNKCQAQHQLEWMSMKEWYQSNNVKEFGISKKELLLAYFLAAATMFEPERTQERIMWAKTQVVSRMITSFLNKENTMSFDLKIALLTQPQHQINGSEMKNGLAQTLPAAFRQLLKEFDKYTRHQLRNTWNKWLMKLKQGDDNGGADAELLANTLNICAGHNEDILSHYEYTALSSLTNKICQRLSQIQDKKMLEIEEGSIKDKEMELEIQTLVKLVLQETSGGIDRNIKQTFLSVFKTFYYRAYHDAKTIDAHIFQVLFEPVV

SEQ ID NO: 4 - protein, 598 aa, Coleus forskohlii
MSSLAGNLRVIPFSGNRVQTRTGILPVHQTPMITSKSSAAVKCSLTTPTDLMGKIKEVFNREVDTSPAAMTTHSTDIPSNLCIIDTLQRLGIDQYFQSEIDAVLHDTYRLWQLKKKDIFSDITTHAMAFRLLRVKGYEVASDELAPYADQERINLQTIDVPTVVELYRAAQERLTEEDSTLEKLYVWTSAFLKQQLLTDAIPDKKLHKQVEYYLKNYHGILDRMGVRRNLDLYDISHYKSLKAAHRFYNLSNEDILAFARQDFNISQAQHQKELQQLQRWYADCRLDTLKFGRDVVRIGNFLTSAMIGDPELSDLRLAFAKHIVLVTRIDDFFDHGGPKEESYEILELVKEWKEKPAGEYVSEEVEILFTAVYNTVNELAEMAHIEQGRSVKDLLVKLWVEILSVFRIELDTWTNDTALTLEEYLSQSWVSIGCRICILISMQFQGVKLSDEMLQSEECTDLCRYVSMVDRLLNDVQTFEKERKENTGNSVSLLQAAHKDERVINEEEACIKVKELAEYNRRKLMQIVYKTGTIFPRKCKDLFLKACRIGCYLYSSGDEFTSPQQMMEDMKSLVYEPLPISPPEANNASGEKMSCVSN

SEQ ID NO: 5 - protein, 587 aa, Coleus forskohlii
MSITINLRVIAFPGHGVQSRQGIFAVMEFPRNKNTFKSSFAVKCSLSTPTDLMGKIKEKLSEKVDNSVAAMATDSADMPTNLCIVDSLQRLGVEKYFQSEIDTVLDDAYRLWQLKQKDIFSDITTHAMAFRLLRVKGYDVSSEELAPYADQEGMNLQTIDLAAVIELYRAAQERVAEEDSTLEKLYVWTSTFLKQQLLAGAIPDQKLHKQVEYYLKNYHGILDRMGVRKGLDLYDAGYYKALKAADRLVDLCNEDLLAFARQDFNINQAQHRKELEQLQRWYADCRLDKLEFGRDVVRVSNFLTSAILGDPELSEVRLVFAKHIVLVTRIDDFFDHGGPREESHKILELIKEWKEKPAGEYVSKEVEILYTAVYNTVNELAERANVEQGRNVEPFLRTLWVQILSIFKIELDTWSDDTALTLDDYLNNSWVSIGCRICILMSMQFIGMKLPEEMLLSEECVDLCRHVSMVDRLLNDVQTFEKERKENTGNAVSLLLAAHKGERAFSEEEAIAKAKYLADCNRRSLMQIVYKTGTIFPRKCKDMFLKVCRIGCYLYASGDEFTSPQQMMEDMKSLVYEPLQIHPPPAN

SEQ ID NO: 6 - protein, 360 aa, Coleus forskohlii
MRSMNLVDAWVQNLPIFKQPHPSKFIHHPRFEPAFLKSRRPISSFAVSAVLTGEEARIFTRGDEAPFNFNAYVVEKATHVNKALDDAVAVKNPPMIHEAMRYSLLAGGKRVRPMLCIAACEVVGGPQAAAIPAACAVEMIHTMSLIHDDLPCMDNDDLRRGKPTNHKVFGENVAVLAGDALLAFAFEFIATATTGVAPERILAAVAELAKAIGTEGLVAGQVVDLHCTGNPNVGLDTLEFIHIHKTAALLEASVVLGAILGGGSSDQVEKLRTFARKIGLLFQVVDDILDVTKSSEELGKTAGKDLAVDKTTYPKLLGLEKAMEFAERLNEEAKQQLLDFDPRKAAPLVALADYIAHRQN

SEQ ID NO: 7 - protein, 722 aa, Coleus forskohlii
MASCGAIGSSFLPLLHSDESSLLSRPTAALHIKKQKFSVGAALYQDNTNDVVPSGEGLTRQKPRTLSFTGEKPSTPILDTINYPIHMKNLSVEELEILADELREEIVYTVSKTGGHLSSSLGVSELTVALHHVFNTPDDKIIWDVGHQAYPHKILTGRRSRMHTIRQTFGLAGFPKRDESPHDAFGAGHSSTSISAGLGMAVGRDLLQKNNHVISVIGDGAMTAGQAYEAMNNAGFLDSNLIIVLNDNKQVSLPTATVDGPAPPVGALSKALTKLQASRKFRQLREAAKGMTKQMGNQAHEIASKVDTYVKGMMGKPGASLFEELGIYYIGPVDGHNIEDLVYIFKKVKEMPAPGPVLIHIITEKGKGYPPAEVAADKMHGVVKFDPTTGKQMKVKTKTQSYTQYFAESLVAEAEQDEKVVAIHAAMGGGTGLNIFQKRFPDRCFDVGIAEQHAVTFAAGLATEGLKPFCTIYSSFLQRGYDQVVHDVDLQKLPVRFMMDRAGLVGADGPTHCGAFDTTYMACLPNMVVMAPSDEAELMHMVATAAVIDDRPSCVRYPRGNGIGVPLPPNNKGIPLEVGKGRILKEGNRVAILGFGTIVQNCLAAAQLLQEHGISVSVADARFCKPLDGDLIKNLVKEHEVLITVEEGSIGGFSAHVSHFLSLNGLLDGNLKWRPMVLPDRYIDHGAYPDQIEEAGLSSKHIAGTVLSLIGGGKDSLHLINM

SEQ ID NO: 8 - protein, 492 aa, Coleus forskohlii
METITLLLALFFIALTYFISSRRRRNLPPGPFPLPIIGNMLQLGSKPHQSFAQLSKKYGPLMSIHLGSLYTVIVSSPEMAKEILQKHGQVFSGRTIAQAVHACDHDKISMGFLPVANTWRDMRKICKEQMFSHHSLEASEELRHQKLQQLLDYAQKCCEAGRAVDIREASFITTLNLMSATMFSTQATEFDSEATKEFKEIIEGVATIVGVANFADYFPILKPFDLQGIKRRADGYFGRLLKLIEGYLNERLESRRLNPDAPRKKDFLETLVDIIEANEYKLTTEHLTHLMLDLFVGGSETNTTSLEWIMSELVINPDKMAKVKEELKSVVGDEKLVNESDMPRLPYLQAVIKEVLRIHPPGPLLLPRKAESDQVVNGYLIPKGTQILFNAWAMGRDPTIWKDPESFEPERFLNQSIDFKGQDFELIPFGSGRRICPGMPLANRILHMTTATLVHNFDWKLEEGTADADHKGELFGLAVRRATPLRIIPLKP

SEQ ID NO: 9 - protein, 495 aa, Coleus forskohlii
MELVQVIAVVAVVVVLWSQLKRKGRKLPPGPSPLPIVGNIFQLSGKNINESFAKLSKIYGPVMSLRLGSLLTVIISSPEMAKEVLTSKDFANRPLTEAAHAHGHSKFSVGFVPVSDPKWKQMRRVCQEEMFASRILENSQQRRHQKLQELIDHVQESRDAGRAVTIRDPVFATTLNIMSLTLFSADATEFSSSATAELRDIMAGVVSVLGAANLADFFPILKYFDPQGMRRKADLHYGRLIDHIKSRMDKRSELKKANPNHPKHDDFLEKIIDITIQRNYDLTINEITHLLVDLYLAGSESTVMTIEWTMAELMLRPESLAKLKAELRSVMGERKMIQESDDISRLPYLNGAIKEALRLHPPGPLLFARKSEIDVELSGYFIPKGTQILVNEWGMGRDPSVWPNPECFQPERFLDKNIDYKGQDPQLIPFGAGRRICPGIPIAHRVVHSVVAALVHNFDWEFAPGGSQCNNEFFTGAALVREVPLKLIPLNPPSI

SEQ ID NO: 10 - protein, 496 aa, Coleus forskohlii
MEFDFPSALIFPAVSLLLLLWLTKTRKPKSDLDRIPGPRRLPLIGNLHHLISLTPPPRLFREMAAKYGPLMRLQLGGVPFLIVSSVDVAKHVVKTNDVPFANRPPMHAARAITYNYTDIGFAPYGEYWRNLRKICTLELLSARRVRSFRHIREEENAGVAKWIASKEGSPANLSERVYLSSFDITSRASIGKATEEKQTLTSSIKDAMKLGGFNVADLYPSSKLLLLITGLNFRIQRVFRKTDRILDDLLSQHRSTSATTERPEDLVDVLLKYQKEETEVHLNNDKIKAVIMDMFLAGGETSATAVDWAMAEMIRNPTTLKKAQEEVRRVFDGKGYVDEEEFHELKYLKLVIKEMLRMHPPLPFLVPRMNSERCEINGYEIPANTRLLINAWAIGRPKYWNDAEKFIPERFENSSIDFKGNNLEYIPFGAGRRMCPGMTFGLASVEFTLAMLLYHFDWKMPQGIKLDMTESFGASLKRKHDLLMIPTLKRPLRLAP

SEQ ID NO: 11 - protein, 500 aa, Coleus forskohlii
MDFFTLLAALFLITLTFFLFFKSESKRRGGANLPPGPYPLPIVGNIFQLGKKPHQSLAQLAKIHGPLMSLHFGSVYTVIVTSPEMAKEIFVKNDQAFLNRTVVEAVHAHDHDKISMAFMDVGTEWRTLRRICKEQMFSTQSLETSQGLRQEKLQQLHDFVQRCCDSGRVVDIREASFVTTLNLMSATLFSIQATEFDSNATEEFREIMEGVASIVGDPNFADYFPILKRFDPQGVKRKAELYFGKMLVLVEDLLQKRQEERRRSPSYAKKDDLLERLVDVLNEKNEYKLTTKHITHLLLDLFVGGSETTTTSVEWIMSELLINPEKLAKLKEELKTVVGEKKQVQESDIPQLPYFEAVLKEVFRLHPPGPLLLPRKAECDVQVGSYTIPKETQILVNAWAIGRDPAIWPNPEAFEPERFLSQKMDYKGQDFELIPFGSGRRICPGLSFANRMLPMTVATLIHNFDWKLEVEANAEDVHKGEMFGIAVRRAVPLRAYPIQP

SEQ ID NO: 12 - protein, 493 aa, Coleus forskohlii
MESMNALVVGLLLIALTILFSLRRRRNLAPGPYPFPIIGNMLQLGTKPHQSFAQLSKKYGPLMSIHLGSLYTVIVSSPEMAKEILQKHGQVFSGRTIAQAVHACDHDKISMGFLPVSNTWRDMRKICKEQMFSHHSLEGSQGLRQQKLLQLLDYAQKCCETGRAVDIREASFITTLNLMSATMFSTQATEFESKSTQEFKEIIEGVATIVGVANFGDYFPILKPFDLQGIKRKADGYFGRLLKLIEGYLNERLESRKSNPNAPRKNDFLETVVDILEANEYKLSVDHLTHLMLDLFVGGSETNTTSLEWTMSELVNNPDKMAKLKQELKSVVGERKLVDESEMPRLPYLQAVIKESLRIHPPGPLLLPRKAETDQEVNGYLIPKGTQILFNVWAMGRDPSIWKDPESFEPERFLNQNIDFKGQDFELIPFGSGRRICPGMPLANRILHMATATMVHNFDWKLEQGTDEADAKGELFGLAVRRAVPLRIIPLQP

SEQ ID NO: 13 - protein, 493 aa, Coleus forskohlii
METMTLLLPLFFIALTYFLSWRRRRNLPPGPFPLPIIGNLLQIGSKPHQSFAQLSKKYGPLMSVQLGSVYTVIASSPEMAKEILQKHGQVFSGRTIAQAAQACGHDQISIGFLPVATTWRDMRKICKEQMFSHHSLESSKELRHEKLQKLLDYAQKCCEAGRAVDIREAAFITTLNLMSATLFSTQATEFDSEATKEFKEVIEGVAVIVGEPNFADYFPILKPFDLQGIKRRANSYFGRLLKLMERYLNERLESRRLNPDAPKKNDFLETLVDIIQADEYKLTTDHVTHLMLDLFVGGSETSATSLEWIMSELVSNPSKLAKVKAELKSVVGEKKVVSESEMARLPYLQAVIKEVLRLHPPGPLLLPRKAGSDQVVNGYLIPKGTQLLFNVWAMGRDPSIWKNPESFEPERFLNQNIDYKGQDFELIPFGSGRRICPGMPLADRIMHMTTATLVHNFDWKLEDGAGDADHKGDDPFGLAIRRATPLRIIPLKP

(19) United States
(12) Patent Application Publication
(10) Pub. No.: US 2019/0194706 A1
(43) Pub. Date: Jun. 27, 2019

(54) Biosynthesis of Oxidised 13R-MO and Related Compounds

(71) Applicant: University of Copenhagen, Copenhagen K (DK)

(72) Inventors: Johan Andersen-Ranberg, Copenhagen (DK); Eirini Pateraki, Vallensbaek Strand (DK)

(21) Appl. No.: 15/523,873

(22) PCT Filed: Nov. 5, 2015
(86) PCT No.: PCT/DK2015/050337

§ 371 Date: May 2, 2017

(30) Foreign Application Priority Data
Nov. 7, 2014 (DK) PA 2014 70682

(51) Int. Cl. C12P 17/06; C07D 311/78

(52) U.S. Cl. CPC C12P 17/06; C12Y 202/01007; C07D 311/78

(57) ABSTRACT

The invention discloses novel methods for biosynthesis of forskolin and other oxidised 13R-MOs. Oxidised 13R-MO may be valuable 011 its own account or as precursors for production of forskolin. In particular, the invention provides methods of producing an oxidised 13R-manoyl oxide (13R-MO) comprising the steps of providing a host organism comprising a heterologous nucleic acid encoding an enzyme capable of catalysing hydroxylation of 13R-MO and or oxidised 13R-MO at the 9 position, wherein said oxidised 13R-MO carries a —H at the 9-position and incubating said host organism in the presence of I3R-MO under conditions allowing growth of said host organism. The invention also discloses materials for use in said methods, in particular the invention provides the enzyme CYP76AH16.

### FIELD OF INVENTION

The present invention relates to the field of biosynthesis of terpenoids. More specifically the invention relates to methods for biosynthesis of oxidised 13R-MO and related compounds, such as to biosynthesis of forskolin.

### BACKGROUND OF INVENTION

Forskolin is a complex functionalised derivative of 13R-MO requiring region- and stereospecific oxidation of five carbon positions. Forskolin is a diterpene naturally produced by Coleus forskohlii. Both Forskolin and oxidized variants of forskolin have been suggested as useful in treatment in a number of clinical conditions. Forskolin has the ability to decrease the intraocular pressure therefore it is used today as an antiglaucoma agent (Wagh K, Patil P, Surana S, Wagh V. Forskolin: Upcoming antiglaucoma molecule, J Postgrad Med 2012, 58(3):199-202), in the form of eye drops. Moreover a water-soluble analogue of forskolin (NKH477) has been approved for commercial use in Japan for treatment of acute heart failure and heart surgery complications because of its vasodilatory effects when administered intravenously (Kikura M, Morita K, Sato S. Pharmacokinetics and a simulation model of colforsin daropate, new forskolin derivative inotropic vasodilator, in patients undergoing coronary artery bypass grafting. Pharmacol Res 2004, 49: 275-281). Forskolin also acts as bronchodilator so it could be used for asthma treatments (Yousif M H and Thulesius O. Forskolin reverses tachyphylaxis to the bronchodilator effects of salbutamol: an in-vitro study on isolated guinea-pig trachea. J Pharm Pharmacol, 1999. 51:181-186). Forskolin may help additionally to treat obesity by contributing to higher rates of body fat burning and promoting lean body mass formation (Godard M P, Johnson B A, Richmond S R. Body composition and hormonal adaptations associated with forskolin consumption in overweight and obese men. Obes Res 2005, 13:1335-1343).

### SUMMARY OF INVENTION

Hitherto forskolin has been purified from Coleus forskohlii or produced chemically. Here novel methods for biosynthesis of forskolin and other oxidised 13R-MO s are presented. Oxidised 13R-MO may be valuable on its own account or as precursors for production of forskolin.

Thus, it is an aspect of the invention to provide methods of producing an oxidised 13R-manoyl oxide (13R-MO), said method comprising the steps of:
a) providing a host organism comprising a heterologous nucleic acid encoding an enzyme capable of catalysing hydroxylation of 13R-MO and/or oxidised 13R-MO at the 9 position, wherein said oxidised 13R-MO carries a —H at the 9-position
b) Incubating said host organism in the presence of 13R-MO under conditions allowing growth of said host organism;
c) Optionally isolating oxidised 13R-MO from the host organism and/or from its surroundings.

If the host organism is a microorganism, then the oxidised 13R-MO may be isolated from the cultivation medium used for cultivation of the host organism.

The host organism may be capable of producing 13R-MO or 13R-MO may be added to the host organism. In preferred embodiments the host organism is capable of producing 13R-MO.

It is also an aspect of the invention to provide host organisms comprising heterologous nucleic acid encoding an enzyme capable of catalysing hydroxylation of 13R-MO and/or oxidised 13R-MO at the 9 position, wherein said oxidised 13R-MO carries a —H at the 9-position.

It is also an aspect of the invention to provide enzymes capable of catalysing hydroxylation of 13R-manoyl oxide (13R-MO) and/or an oxidised 13R-MO at the 9 position, wherein said oxidised 13R-MO carries a —H at the 9-position.

### DESCRIPTION OF DRAWINGS

FIG. 1 shows LC-MS analysis of extracts from assays with Nicotiana benthamiana transiently expressing CfTPS2, CfTPS3, CYP76AH8, CYP76AH11 and CYP76AH16 from Coleus forskohlii (upper panel), and Nicotiana benthamiana transiently expressing CfGGPPS, CfDXS, CfTPS2, CfTPS3, CYP76AH8, CYP76AH11 and CYP76AH16 from Coleus forskohlii (lower panel). LC-MS spectrum with retention time in minutes is shown for the extract and for a deacetyl-forskolin standard.

FIG. 2 shows an overview of the biosynthesis of forskolin. Each column shows the compounds produced by CYP76AH8, CYP76AH11 and CYP76AH16 separately or in combination as indicated. The right hand column shows the structure of the compounds as well as one route to forskolin. The left hand column indicates the chemical formulae of the compounds. The numbers in the table are the same compound numbers used in FIG. 4.

FIG. 3 shows 13R-manoyl-oxide and oxidised 13R-manoyl-oxide found in C. forskohlii exhibiting pharmaceutical properties.

FIG. 4 shows selected oxidation reactions of 13R-MO en route to forskolin and other oxidised 13R-MO compounds indicating enzymes involved in the various reactions. * indicates possible position of —OH group(s). Similar reactions are observed if CYP76AH8 is exchanged with either CYP76AH15 or CYP76AH17.

FIG. 5 shows a proposed biosynthetic route to forskolin in C. forskohlii proposed by Asada et al., Phytochemistry 79 (2012) 141-146.

FIG. 6 shows GC-MS analysis of extracts from N. benthamiana transiently expressing CfCXS, CfGGPPs, CfTPS2, CfTPS3 and p19 in combination with water (−), CYP76AH15, CYP76AH17, CYP76AH8, CYP76AH11 or CYP76AH16, respectively.

FIG. 7 shows an overview of compounds identified by LC-MS-qTOF in extracts from N. benthamiana transiently expressing CfCXS, CfGGPPs, CfTPS2 and CfTPS3 in combination with water (−) or CYP76AH15, CYP76AH11 and CYP76AH16 (I) or CYP76AH17, CYP76AH11 and CYP76AH16 (II) or CYP76AH8, CYP76AH11 or CYP76AH16 (III), respectively.

FIG. 8 shows an overview of CYP76AH′s involved in the oxygenation of 13R-manoyl oxide (MO) for the production of forskolin.

### DETAILED DESCRIPTION OF THE INVENTION

### Methods of Preparing Oxidised 13R-MO

It is one aspect of the present invention to provide biosynthetic methods for preparing oxidised 13R-MO. The methods of the invention generally comprise the steps of:
a) Providing a host organism comprising a heterologous nucleic acid encoding an enzyme capable of catalysing hydroxylation of 13R-MO and/or oxidised 13R-MO at the 9 position, wherein said oxidised 13R-MO carries a —H at the 9-position,
b) Incubating said host organism in the presence of 13R-MO under conditions allowing growth of said host organism
c) Optionally isolating oxidised 13R-MO from the host organism.

The structure of 13R-MO is provided herein below in the section “Oxidised 13R-MO.

Said enzyme capable of catalysing hydroxylation of 13R-MO and/or oxidised 13R-MO at the 9 position may be any of the enzymes described herein below in the section “Enzyme catalysing hydroxylation of 13R-MO and/or oxidised 13R-MO at the 9 position”.

The oxidised 13R-MO may be any of the oxidised 13R-MO described herein below in the section “Oxidised 13R-MO”.

The term “oxidised 11-hydroxyl-13R-MO” as used herein refers to 11-hydroxyl-13R-MO further substituted at one or more of the positions 1, 6, 7 and 9 with a moiety selected from the group consisting of ═O, —OH and OR, wherein R preferably is acyl.

The term “oxidised hydroxylated-13R-MO” as used herein refers to 13R-MO, which is substituted with hydroxyl on at least one of the positions 1, 6, 7 and 9, and which further is substituted at one or more of the others positions 1, 6, 9 and 11 with a moiety selected from the group consisting of —O, —OH and OR, wherein R preferably is acyl.

The term “oxidised 11-keto-13R-MO” as used herein refers to 13R-MO, which is substituted with oxo at the 11 position and which further is substituted at one or more of the positions 1, 6, 9 and 11 with a moiety selected from the group consisting of ═O, —OH and OR, wherein R preferably is acyl.

In addition to the heterologous nucleic acid encoding an enzyme capable of catalysing hydroxylation of 13R-MO and/or oxidised 13R-MO at the 9 position, the host organism may also comprise one or more of the following heterologous nucleic acids:
I. A heterologous nucleic acid encoding an enzyme capable of catalysing hydroxylation of 13R-manoyl oxide (13R-MO) and/or an oxidised 13R-MO derivative at the 11 position, wherein said oxidised 13R-MO carries a —H at the 11-position; and/or catalysing oxidation of the hydroxyl group to form an oxo-group at the 11 position of 11-hydroxyl-13R-MO and/or an oxidised 11-hydroxyl-13R-MO. The heterologous nucleic acid I. may for example be a heterologous encoding any of the enzymes described in the section “I. Enzyme catalysing hydroxylation of 13R-MO at the 11 position” herein below.
II. A heterologous nucleic acid encoding an enzyme capable of catalysing hydroxylation of 13R-MO and/or oxidised 13R-MO at the 1 position, wherein said oxidised 13R-MO carries a —H at the 1-position. The heterologous nucleic acid II. may for example be a heterologous encoding any of the enzymes described in the section “II. Enzyme catalysing hydroxylation of 13R-MO at the 1 position” herein below.
III. A heterologous nucleic acid encoding an enzyme capable of catalysing hydroxylation of 13R-MO and/or oxidised 13R-MO at the 6 position, wherein said oxidised 13R-MO carries a —H at the 6-position. The heterologous nucleic acid III. may for example be a heterologous encoding any of the enzymes described in the section “III. Enzyme catalysing hydroxylation of 13R-MO at the 6 position” herein below.
IV. A heterologous nucleic acid encoding an enzyme capable of catalysing hydroxylation of 13R-MO and/or oxidised 13R-MO at the 7 position, wherein said oxidised 13R-MO carries a —H at the 7-position The heterologous nucleic acid IV. may for example be a heterologous encoding any of the enzymes described in the section “IV. Enzyme catalysing hydroxylation of 13R-MO at the 7 position” herein below.
V. A heterologous nucleic acid encoding an enzyme capable of catalysing transfer of an acyl group to an —OH of a hydroxylated 13R-MO and/or an oxidised hydroxylated-13R-MO. The heterologous nucleic acid V. may for example be a heterologous encoding any of the enzymes described in the section “V. Enzyme catalysing transfer of an acyl group” herein below.
VI. A heterologous nucleic acid encoding TPS2, such as any of the TPS2 described herein below in the section “VI. TPS2” herein below.
VII. A heterologous nucleic acid encoding TPS3, such as any of the TPS3 described herein below in the section “VII. TPS3” herein below.
VIII. A heterologous nucleic acid encoding TPS4, such as any of the TPS4 described herein below in the section “VIII. TPS4” herein below.
IX. A heterologous nucleic acid encoding an enzyme involved in the synthesis of GGPP, such as any of the enzymes described herein below in the section “IX. GGPP herein below
X. A heterologous nucleic acid encoding a 1-deoxy-D-xylulose-5-phosphate synthase, such as any of the 1-deoxy-D-xylulose-5-phosphate synthases described herein below in the section “X. 1-deoxy-D-xylulose-5-phosphate synthase” herein below.

The host organism may comprise one of more of the heterologous nucleic acids I., II., III., IV., V., VI, VII, VIII, IX and X, such as at least 2, for example at least 3, such as at least 4, for example at least 5, such as all of heterologous nucleic acids I., II., III., IV., V., VI, VII VIII, IX and X.

Incubating said host organism in the presence of 13R-MO may be obtained in several manners. For example, 13R-MO may be added to the host organism. If the host organism is a microorganism, then 13R-MO may be added to the cultivation medium of said microorganism. If the host organism is a plant, then 13R-MO may be added to the growing soil of the plant or it may be introduced into the plant by infiltration. Thus, if the heterologous nucleic(s) are introduced into the plant by infiltration, then 13R-MO may be co-infiltrated together with the heterologous nucleic acid(s).

It is also comprised within the invention that the host organism is capable of producing 13R-MO. In such embodiments incubating said host organism in the presence of 13R-MO simply requires cultivating said host organism.

In preferred embodiments of the invention the host organism comprises in addition to the heterologous nucleic acid encoding an enzyme capable of catalysing hydroxylation of 13R-MO and/or oxidised 13R-MO at the 9 position at least the following heterologous nucleic acids:
VI. A heterologous nucleic acid encoding TPS2, such as any of the TPS2 described herein below in the section VI. TPS2
VII. A heterologous nucleic acid encoding TPS3, such as any of the TPS3 described herein below in the section VII, and/or
VIII. A heterologous nucleic acid encoding TPS4, such as any of the TPS4 described herein below in the section VIII

Such host organisms are in general capable of producing 13R-MO and thus, no 13R-MO needs to be added to such host organisms. In such embodiments it is preferable that the host organism is incubated in the presence of GGPP. Many host organisms are capable of producing GGPP, and thus incubation in the presence of GGPP may be simply require cultivation of the host organism.

In other preferred embodiments of the invention the host organism comprises in addition to the heterologous nucleic acid encoding an enzyme capable of catalysing hydroxylation of 13R-MO and/or oxidised 13R-MO at the 9 position at least the following heterologous nucleic acids:
VI. A heterologous nucleic acid encoding TPS2, such as any of the TPS2 described herein below in the section VI. TPS2
VII. A heterologous nucleic acid encoding TPS3, such as any of the TPS3 described herein below in the section VII, and/or
VIII. A heterologous nucleic acid encoding TPS4, such as any of the TPS4 described herein below in the section VIII
IX. A heterologous nucleic acid encoding a enzyme involved in the synthesis of GGPP, such as any of the enzymes described herein below in the section IX
X. A heterologous nucleic acid encoding a 1-deoxy-D-xylulose-5-phosphate synthase, such as any of the 1-deoxy-D-xylulose-5-phosphate synthases described herein below in the section “X. 1-deoxy-D-xylulose-5-phosphate synthase ” herein below.

Such host organisms are in general capable of producing 13R-MO and GGPP, and thus incubation in the presence of GGPP and 13R-MO simply require cultivation of the host organism.

The methods of the invention may also be performed in vitro. Thus, the method of producing an oxidised 13R-MO may comprise the steps of
a) providing a host organism comprising a heterologous nucleic acid encoding an enzyme capable of catalysing hydroxylation of 13R-MO and/or oxidised 13R-MO at the 9 position and optionally one or more of the heterologous nucleic acids I., II., III., IV., V., VI and VII or one or more of the heterologous nucleic acids I., II., III., IV., V., VI., VII., VIII, IX and X, and preferably comprising at least the heterologous nucleic acid I.,
b) preparing an extract of said host organism;
c) providing 13R-MO
d) incubating said extract with 13R-MO

thereby producing oxidised 13R-MO.

The host organism may be any of the host organisms described herein below in the section “Host organism”.

Enzyme Catalysing Hydroxylation of 13R-MO and/or Oxidised 13R-MO at the 9 Position

The host organisms to be used with the present invention comprise a heterologous nucleic acid encoding an enzyme capable of catalysing hydroxylation of 13R-MO and/or oxidised 13R-MO at the 9 position, wherein said oxidised 13R-MO carries a —H at the 9-position.

Said enzyme may for example be any of the enzymes described herein in this section and may also be referred to herein as “enzyme capable of catalysing hydroxylation of 13R-MO and/or oxidised 13R-MO at the 9 position”.

It is preferred that said enzyme is capable of catalysing the following reaction A:

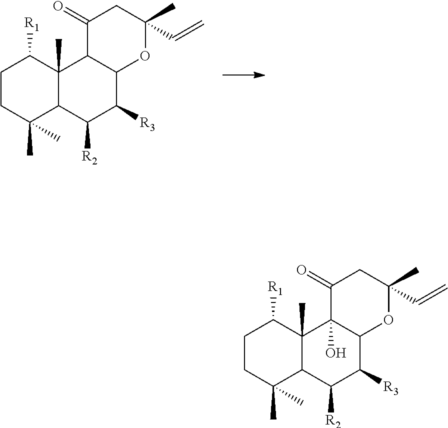

wherein R₁, R₂ and R₃ individually are selected from the group consisting of —H, —OH and —OR, wherein R preferably is acyl. Acyl is as defined in the section “Oxidised 13R-MO” herein below.

In particular, R₁, R₂ and R₃ may individually be selected from the group consisting of —H, and —OH. Thus, at least one of R₁, R₂ and R₃ may be —H, for example at least two of R₁, R₂ and R₃ may be —H, for example all of R₁, R₂ and R₃ may be —H. Similarly, at least one of R₁, R₂ and R₃ may be —OH, for example at least two of R₁, R₂ and R₃ may be —OH, for example all of R₁, R₂ and R₃ may be —OH.

It is also preferred that said enzyme is capable of catalysing the following reaction B:

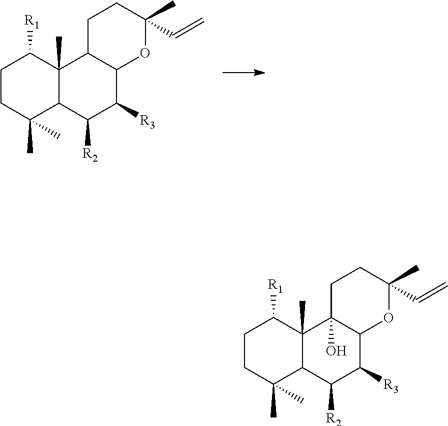

wherein R₁, R₂ and R₃ individually are selected from the group consisting of —H, —OH and —OR, wherein R preferably is acyl. Acyl is as defined in the section “Oxidised 13R-MO” herein below.

In particular, R₁, R₂ and R₃ may individually be selected from the group consisting of —H, and —OH. Thus, at least one of R₁, R₂ and R₃ may be —H, for example at least two of R₁, R₂ and R₃ may be —H, for example all of R₁, R₂ and R₃ may be —H. Similarly, at least one of R₁, R₂ and R₃ may be —OH, for example at least two of R₁, R₂ and R₃ may be —OH, for example all of R₁, R₂ and R₃ may be —OH.

Thus, the enzyme capable of catalysing hydroxylation of 13R-MO and/or oxidised 13R-MO at the 9 position may be capable of catalysing reaction A or reaction B or preferably both of reactions A and B outlined above.

In particular, it is preferred that the enzyme capable of catalysing hydroxylation of 13R-MO and/or oxidised 13R-MO at the 9 position is capable of catalysing at least some of the following reactions:
1) Reaction A, wherein R₁, R₂ and R₃ are —H
2) Reaction A, wherein R₁ is —OH and R₂ and R₃ are —H
3) Reaction A, wherein R₁ and R₂ are —OH and R₃ is —H
4) Reaction A, wherein R₁ is —H and R₂ and R₃ are —OH
5) Reaction A, wherein R₁ and R₂ are —H and R₃ are —OH
6) Reaction A, wherein R₁, R₂ and R₃ are —OH
7) Reaction B, wherein R₁, R₂ and R₃ are —H
8) Reaction B, wherein R₁ is —OH and R₂ and R₃ are —H
9) Reaction B, wherein R₁ and R₂ are —OH and R₃ is —H
10) Reaction B, wherein R₁ is —H and R₂ and R₃ are —OH
11) Reaction B, wherein R₁ and R₂ are —H and R₃ are —OH
12) Reaction B, wherein R₁, R₂ and R₃ are —OH

For example the enzyme capable of catalysing hydroxylation of 13R-MO and/or oxidised 13R-MO at the 9 position is capable of catalysing at least two, such as at least 5, for example at least 10, such as all of the reactions 1)-12) listed above,

In particular, it is preferred that the enzyme capable of catalysing hydroxylation of 13R-MO and/or oxidised 13R-MO at the 9 position is capable of catalysing at least the following two reactions:
6) Reaction A, wherein R₁, R₂ and R₃ are —OH
7) Reaction B, wherein R₁, R₂ and R₃ are —H

The enzyme capable of catalysing hydroxylation of 13R-MO and/or oxidised 13R-MO at the 9 position may be any useful enzyme with above mentioned activities, in particular said enzyme may be a CYP450. As used herein the term “CYP450” is used to refer to cytochrome P450. CYP450 is also known as P450 or CYP. The enzyme capable of catalysing hydroxylation of 13R-MO and/or oxidised 13R-MO at the 9 position may be derived from any suitable source, but in a preferred embodiment said enzyme is an enzyme from Coleus forskohlii. Thus the enzyme capable of catalysing hydroxylation of 13R-MO and/or oxidised 13R-MO at the 9 position may be a CYP450 from Coleus forskohlii.

It is preferred that the enzyme capable of catalysing hydroxylation of 13R-MO and/or oxidised 13R-MO at the 9 position is CYP76AH16. Thus preferably the enzyme capable of catalysing hydroxylation of 13R-MO and/or oxidised 13R-MO at the 9 position may be CYP76AH16 of SEQ ID NO:2 and functional homologues of any of the aforementioned sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

Thus, in a preferred embodiment of the invention the enzyme capable of catalysing hydroxylation of 13R-MO and/or oxidised 13R-MO at the 9 position is capable of catalysing the following reaction C:

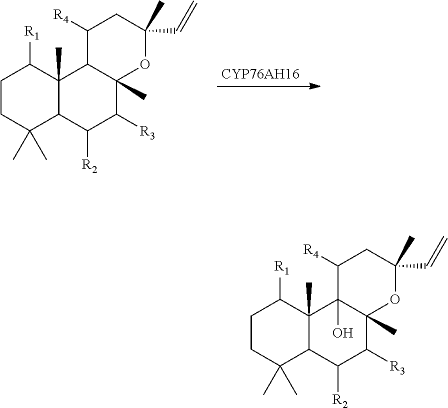

wherein R₁ is H or OH,

R₂ is H or OH,

R₃ is H or OH, and

R₄ is H, OH or ═O

The sequence identity is preferably calculated as described herein below in the section “Sequence identity”. A functional homologue of CYP76AH16 may be capable of catalysing reactions A and/or B described above

The heterologous nucleic acid encoding the enzyme capable of catalysing hydroxylation of 13R-MO and/or oxidised 13R-MO at the 9 position may be any heterologous nucleic acid encoding any of the enzymes with said activity described herein. Thus, the heterologous nucleic acid may be any heterologous nucleic acid encoding CYP76AH16 of SEQ ID NO:2 or any of the functional homologues thereof described herein above. In one embodiment the heterologous nucleic acid may comprise or consist of SEQ ID NO:1.

I. Enzyme Catalysing Hydroxylation of 13R-MO at the 11 Position

In addition to the heterologous nucleic acid encoding the enzyme capable of catalysing hydroxylation of 13R-MO and/or oxidised 13R-MO at the 9 position, the host organism may comprise a heterologous nucleic acid encoding an enzyme capable of
a) catalysing hydroxylation of 13R-manoyl oxide (13R-MO) and/or an oxidised 13R-MO derivative at the 11 position, wherein said oxidised 13R-MO carries a —H at the 11-position; and/or
b) catalysing oxidation of the hydroxyl group at the 11 position of 11-hydroxyl-13R-MO and/or an oxidised 11-hydroxyl-13R-MO to form an oxo-group.

Said enzyme may for example be any of the enzymes described herein in this section and may also be referred to herein as “enzyme I”. It is in particular preferred that the host organism comprises a heterologous nucleic acid encoding said enzyme, in embodiments of the invention, wherein the oxidised 13R-MO to be produced is substituted at the 11 position with a moiety selected from the group consisting of ═O, —OH and OR, wherein R preferably is acyl, and in particular in embodiments of the invention, wherein the oxidised 13R-MO to be produced is substituted at the 11 position with oxo (═O).

The enzyme I may be an enzyme having one or two functions. In particular it is preferred that the enzyme I is capable of catalysing the following reaction Ia:

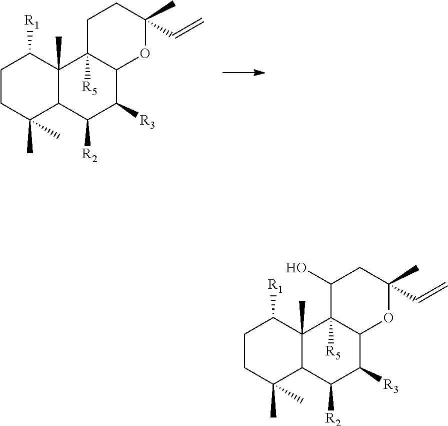

wherein R₁, R₂, R₃ and R₅ individually are selected from the group consisting of —H, —OH and —OR, wherein R preferably is acyl. Acyl is as defined in the section “Oxidised 13R-MO” herein below.

In particular, at least one of R₁, R₂, R₃ and R₅ is —H, for example at least two of R₁, R₂, R₃ and R₅ is —H, for example at least three of R₁, R₂, R₃ and R₅ is —H. In one embodiment all of R₁, R₂, R₃ and R₅ is —H.

It is also preferred that the enzyme I is capable of catalysing the following reaction Ib:

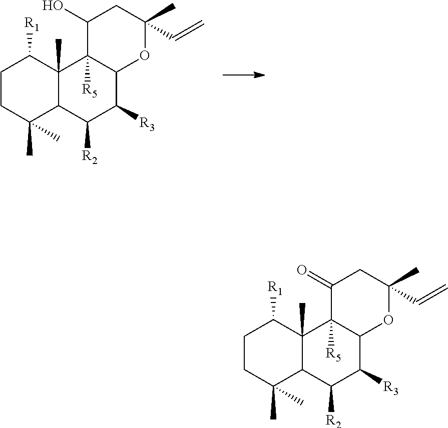

wherein R₁, R₂, R₃ and R₅ individually are selected from the group consisting of —H, —OH and —OR, wherein R preferably is acyl. Acyl is as defined in the section “Oxidised 13R-MO” herein below.

In particular, at least one of R₁, R₂, R₃ and R₅ is —H, for example at least two of R₁, R₂, R₃ and R₅ is —H, for example at least three of R₁, R₂, R₃ and R₅ is —H. In one embodiment all of R₁, R₂, R₃ and R₅ is —H.

It is even more preferred that enzyme I is capable of catalysing both of reactions Ia and Ib outlined above.

It is also possible that enzyme I is capable of catalysing the reaction Ic:

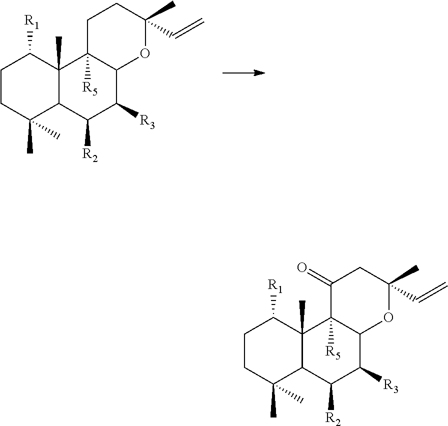

In particular, at least one of R₁, R₂, R₃ and R₅ is —H, for example at least two of R₁, R₂, R₃ and R₅ is —H, for example at least three of R₁, R₂, R₃ and R₅ is —H. In one embodiment all of R₁, R₂, R₃ and R₅ is —H.

Enzyme I may be any useful enzyme with above mentioned activities, in particular enzyme I may be a CYP450. Enzyme I may be derived from any suitable source, but in a preferred embodiment enzyme I is an enzyme from Coleus forskohlii. Thus enzyme I may be a CYP450 from Coleus forskohlii.

In a preferred embodiment of the invention, enzyme I is CYP76AH8. Thus, enzyme I may be CYP76AH8 from Coleus forskohlii. In particular, enzyme I may be CYP76AH8 of SEQ ID NO:8 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith. Functional homologues of CYP76AH8 may be enzymes sharing aforementioned sequence identity with SEQ ID NO:8, and which also are capable of catalysing reaction(s) described in this section.

In one embodiment of the invention, enzyme I is CYP76AH17. Thus, enzyme I may be CYP76AH17 from Coleus forskohlii. In particular, enzyme I may be CYP76AH17 of SEQ ID NO:12 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

Functional homologues of CYP76AH17 may be enzymes sharing aforementioned sequence identity with SEQ ID NO:12, and which also are capable of catalysing reaction(s) described in this section.

In one embodiment of the invention, enzyme I is CYP76AH15. Thus, enzyme I may be CYP76AH15 from Coleus forskohlii. In particular, enzyme I may be CYP76AH15 of SEQ ID NO:13 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith. Functional homologues of CYP76AH15 may be enzymes sharing aforementioned sequence identity with SEQ ID NO:13, and which also are capable of catalysing reaction(s) described in this section.

In preferred embodiments of the invention, the host organism comprise a heterologous nucleic acid encoding enzyme I, wherein enzyme I is selected from the group consisting of CYP76AH8 of SEQ ID NO:8, CYP76AH17 of SEQ ID N012, CYP76AH15 of SEQ ID NO:13 and any of the functional homologues of the aforementioned described herein above. Very preferably enzyme I may be CYP76AH15 of SEQ ID NO:13 and any of the functional homologues thereof described herein above.

In embodiments of the invention, wherein enzyme I catalyses reaction I, then enzyme I may be CYP76AH11. Thus, enzyme I may be CYP76AH11 from Coleus forskohlii. In particular, enzyme I may be CYP76AH11 of SEQ ID NO:9 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith. Functional homologues of CYP76AH11 may be enzymes sharing aforementioned sequence identity with SEQ ID NO:8, and which also are capable of catalysing reaction I.

The heterologous nucleic acid encoding the enzyme I may be any heterologous nucleic acid encoding any of the enzyme Is described herein in this section.

Thus, in one embodiment the heterologous nucleic acid may be any heterologous nucleic acid encoding CYP76AH8 of SEQ ID NO:8 or any of the functional homologues thereof described herein above. In one embodiment the heterologous nucleic acid may comprise or consist of the sequence described in international patent application WO2015113569 as SEQ ID NO:5.

In one embodiment the heterologous nucleic acid may be any heterologous nucleic acid encoding CYP76AH11 of SEQ ID NO:9 or any of the functional homologues thereof described herein above. In one embodiment the heterologous nucleic acid may comprise or consist of the sequence described in international patent application WO2015113569 as SEQ ID NO:6.

In one embodiment the heterologous nucleic acid may be any heterologous nucleic acid encoding CYP76AH15 of SEQ ID NO:13 or any of the functional homologues thereof described herein above. In one embodiment the heterologous nucleic acid may comprise or consist of the sequence described in international patent application WO2015113569 as SEQ ID NO:12.

In one embodiment the heterologous nucleic acid may be any heterologous nucleic acid encoding CYP76AH17 of SEQ ID NO:12 or any of the functional homologues thereof described herein above. In one embodiment the heterologous nucleic acid may comprise or consist of the sequence described in international patent application WO02015113569 as SEQ ID NO:13.

II. Enzyme Catalysing Hydroxylation of 13R-MO at the 1 Position

In addition to the heterologous nucleic acid encoding the enzyme capable of catalysing hydroxylation of 13R-MO and/or oxidised 13R-MO at the 9 position, the host organism may comprise a heterologous nucleic acid encoding an enzyme capable of catalysing hydroxylation of 13R-MO and/or oxidised 13R-MO at the 1 position, wherein said oxidised 13R-MO carries a —H at the 1-position. For example, said enzyme may be capable of catalysing hydroxylation of oxidised 11-keto-13R-MO at the 1 position.

Said enzyme may for example be any of the enzymes described herein in this section and may also be referred to herein as “enzyme II”. It is in particular preferred that the host organism comprises a heterologous nucleic acid encoding said enzyme, in embodiments of the invention, wherein the oxidised 13R-MO to be produced is substituted at least at the 1 position with a moiety selected from the group consisting of —OH and OR, wherein R preferably is acyl.

It is preferred that the enzyme II is capable of catalysing the following reaction IIa:

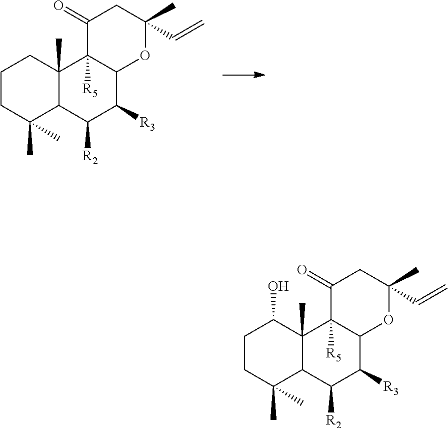

wherein R₂, R₃ and R₅ individually are selected from the group consisting of —H, —OH and —OR, wherein R preferably is acyl. Acyl is as defined in the section “Oxidised 13R-MO” herein below.

In particular, at least one of R₂, R₃ and R₅ is —H, for example at least two of R₂, R₃ and R₅ is —H, for example all of R₂, R₃ and R₅ is —H.

In one preferred embodiment enzyme II is capable of catalysing reaction IIa, wherein R₂ and R₃ is —OH and R₅ is —H.

It is also preferred that enzyme II is capable of catalysing the following reaction IIb:

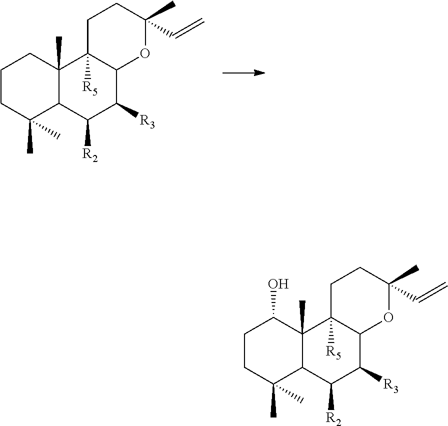

wherein R₂, R₃ and R₅ individually are selected from the group consisting of —H, —OH and —OR, wherein R preferably is acyl. acyl is as defined in the section “Oxidised 13R-MO” herein below.

In particular, at least one of R₂, R₃ and R₅ is —H, for example at least two of R₂, R₃ and R₅ is —H, for example all of R₂, R₃ and R₅ is —H.

Thus, enzyme II may be capable of catalysing reaction IIa or reaction IIb or both of reactions IIa and IIb outlined above. It is also comprised within the invention that said enzyme in addition to being able to catalyse reactions IIa and/or IIb outlined above also may be able to catalyse other reactions, e.g. reactions IIIa, IIIb, IVa, IVb, Va or Vb outlined below.

Enzyme II may be any useful enzyme with above mentioned activities, in particular enzyme II may be a CYP450. Enzyme II may be derived from any suitable source, but in a preferred embodiment enzyme II is an enzyme from Coleus forskohlii. Thus enzyme II may be a CYP450 from Coleus forskohlii.

In one embodiment of the invention, enzyme II is selected from the group consisting of CYP76AH11, CYP71 D381 and CYP76AH9. Thus, enzyme II may be CYP76AH11 of SEQ ID NO:9, CYP71 D381 of SEQ ID NO:10, CYP76AH9 of SEQ ID NO:11 or a functional homologue of any of the aforementioned sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

In particular, in embodiments of the invention wherein enzyme II is capable of catalysing reaction IIa, then enzyme II may be CYP76AH11.Thus, enzyme II may be CYP76AH11 in embodiments of the invention, wherein enzyme II is capable of catalysing reaction II, wherein R₂ and R₃ is —OH and R₅ is —H. Thus, enzyme II may be CYP76AH11 of SEQ ID NO:9 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith. Functional homologues of CYP76AH11 may be enzymes sharing aforementioned sequence identity with SEQ ID NO:9, and which also are capable of catalysing reaction II.

In embodiments of the invention wherein enzyme II is capable of catalysing reaction IIb, then enzyme II may in particular be CYP71 D381 or CYP76AH9. Thus, enzyme II may be CYP71 D381 of SEQ ID NO:10 or CYP76AH9 of SEQ ID NO:11 or any of the functional homologues thereof described herein above.

The heterologous nucleic acid encoding the enzyme II may be any heterologous nucleic acid encoding any of the enzyme IIs described herein in this section. Thus, in one embodiment the heterologous nucleic acid may be any heterologous nucleic acid encoding CYP76AH11 of SEQ ID NO:9 or any of the functional homologues thereof described herein above. In one embodiment the heterologous nucleic acid may comprise or consist of the sequence described in international patent application WO2015113569 as SEQ ID NO:6.

III. Enzyme Catalysing Hydroxylation of 13R-MO at the 6 Position

In addition to the heterologous nucleic acid encoding the enzyme capable of catalysing hydroxylation of 13R-MO and/or oxidised 13R-MO at the 9 position, the host organism may comprise a heterologous nucleic acid encoding an enzyme capable of catalysing hydroxylation of 13R-MO and/or oxidised 13R-MO at the 6 position, wherein said oxidised 13R-MO carries a —H at the 6-position. For example, said enzyme may be capable of catalysing hydroxylation of oxidised 11-keto-13R-MO at the 6 position.

Said enzyme may for example be any of the enzymes described herein in this section and may also be referred to herein as “enzyme III”. It is in particular preferred that the host organism comprises a heterologous nucleic acid encoding said enzyme, in embodiments of the invention, wherein the oxidised 13R-MO to be produced is substituted at least at the 6 position with a moiety selected from the group consisting of —OH and OR, wherein R preferably is acyl.

It is preferred that the enzyme III is capable of catalysing the following reaction IIIa:

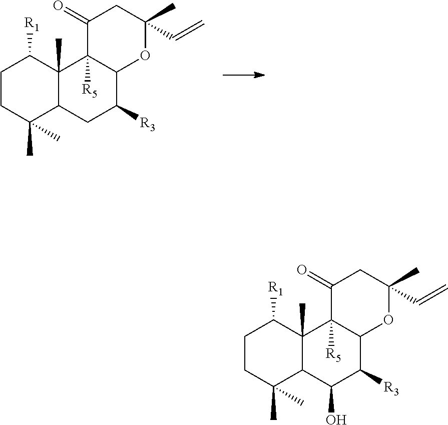

wherein R₁, R₃ and R₅ individually are selected from the group consisting of —H, —OH and —OR, wherein R preferably is acyl. Acyl is as defined in the section “Oxidised 13R-MO” herein below.

In particular, at least one of R₁, R₃ and R₅ is —H, for example at least two of R₁, R₃ and R₅ is —H, for example all of R₁, R₃ and R₅ is —H.

It is also preferred that enzyme III is capable of catalysing the following reaction IIIb:

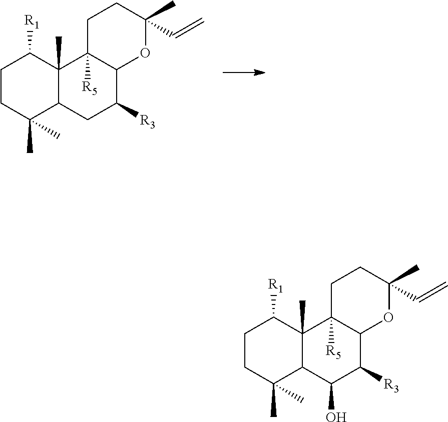

wherein R₁, R₃ and R₅ individually are selected from the group consisting of —H, —OH and —OR, wherein R preferably is acyl. acyl is as defined in the section “Oxidised 13R-MO” herein below.

In particular, at least one of R₁, R₃ and R₅ is —H, for example at least two of R₁, R₃ and R₅ is —H, for example all of R₁, R₃ and R₅ is —H.

In one preferred embodiment enzyme III is capable of catalysing reaction IIIa, wherein all of R₁, R₃ and R₅ are —H.

Thus, enzyme III may be capable of catalysing reaction IIIa or reaction IIIb or both of reactions IIIa and IIIb outlined above. It is also comprised within the invention that said enzyme in addition to being able to catalyse reactions IIIa and/or IIIb outlined above also may be able to catalyse other reactions, e.g. reactions IIa, IIb, IVa, IVb, Va or Vb outlined herein.

Enzyme III may be any useful enzyme with above mentioned activities, in particular enzyme III may be a CYP450. Enzyme III may be derived from any suitable source, but in a preferred embodiment enzyme III is an enzyme from Coleus forskohlii. Thus enzyme III may be a CYP450 from Coleus forskohlii.

In one embodiment of the invention, enzyme III is selected from the group consisting of CYP76AH11, CYP71 D381 and CYP76AH9. Thus, enzyme III may be CYP76AH11 of SEQ ID NO:9, CYP71 D381 of SEQ ID NO:10, CYP76AH9 of SEQ ID NO:11 or a functional homologue of any of the aforementioned sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

In particular, in embodiments of the invention wherein enzyme III is capable of catalysing reaction IIIa, then enzyme III may be CYP76AH11. Thus, enzyme III may be CYP76AH11 of SEQ ID NO:9 or any of the functional homologues thereof described herein above also capable of catalysing reaction IIIa.

In embodiments of the invention wherein enzyme III is capable of catalysing reaction 111b, then enzyme III may in particular be CYP71 D381 or CYP76AH9. Thus, enzyme III may be CYP71 D381 of SEQ ID NO:10 or CYP76AH9 of SEQ ID NO:11 or any of the functional homologues thereof described herein above.

The heterologous nucleic acid encoding the enzyme III may be any heterologous nucleic acid encoding any of the enzyme Ills described herein in this section. Thus, in one embodiment the heterologous nucleic acid may be any heterologous nucleic acid encoding CYP76AH11 of SEQ ID NO:9 or any of the functional homologues thereof described herein above. In one embodiment the heterologous nucleic acid may comprise or consist of the sequence described in international patent application WO2015113569 as SEQ ID NO:6.

IV. Enzyme Catalysing Hydroxylation of 13R-MO at the 7 Position

In addition to the heterologous nucleic acid encoding the enzyme capable of catalysing hydroxylation of 13R-MO and/or oxidised 13R-MO at the 9 position, the host organism may comprise a heterologous nucleic acid encoding an enzyme capable of catalysing hydroxylation of 13R-MO and/or oxidised 13R-MO at the 7 position, wherein said oxidised 13R-MO carries a —H at the 7-position. For example, said enzyme may be capable of catalysing hydroxylation of oxidised 11-keto-13R-MO at the 7 position.

Said enzyme may for example be any of the enzymes described herein in this section and may also be referred to herein as “enzyme IV”. It is in particular preferred that the host organism comprises a heterologous nucleic acid encoding said enzyme, in embodiments of the invention, wherein the oxidised 13R-MO to be produced is substituted at least at the 7 position with a moiety selected from the group consisting of —OH and OR, wherein R preferably is acyl.

It is preferred that the enzyme IV is capable of catalysing the following reaction IVa:

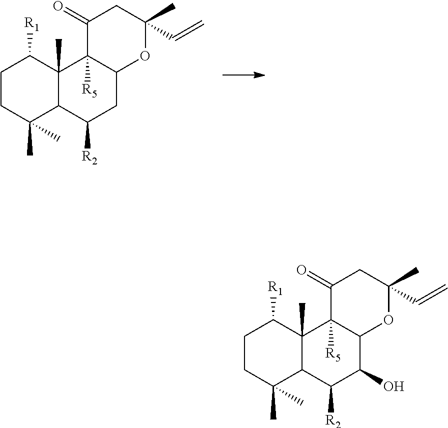

wherein R₁, R₂ and R₅ individually are selected from the group consisting of —H, —OH and —OR, wherein R preferably is acyl. acyl is as defined in the section “Oxidised 13R-MO” herein below.

In particular, at least one of R₁, R₂ and R₅ is —H, for example at least two of R₁, R₂ and R₅ is —H, for example all of R₁, R₂ and R₅ is —H.

In one preferred embodiment enzyme IV is capable of catalysing reaction IVa, wherein R_{I} and R₅ are —H and R₂ is —OH.

It is also preferred that enzyme IV is capable of catalysing the following reaction IVb:

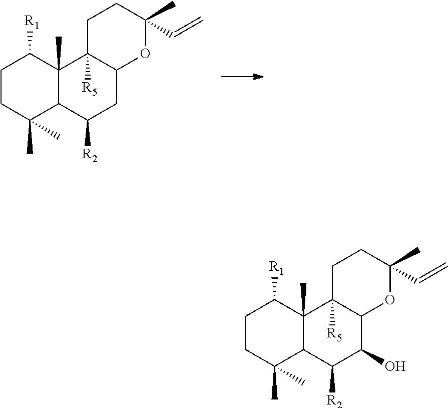

wherein R₁, R₂ and R₅ individually are selected from the group consisting of —H, —OH and —OR, wherein R preferably is acyl. acyl is as defined in the section “Oxidised 13R-MO” herein below.

In particular, at least one of R₁, R₂ and R₅ is —H, for example at least two of R₁, R₂ and R₅ is —H, for example all of R₁, R₂ and R₅ is —H.

Thus, enzyme IV may be capable of catalysing reaction IVa or reaction IVb or both of reactions IVa and IVb outlined above. It is also comprised within the invention that said enzyme in addition to being able to catalyse reactions IVa and/or IVb outlined above also may be able to catalyse other reactions, e.g. reactions Iaa, Ib, IIa, IIb, Va or Vb outlined herein.

Enzyme IV may be any useful enzyme with above mentioned activities, in particular enzyme IV may be a CYP450. Enzyme IV may be derived from any suitable source, but in a preferred embodiment enzyme III is an enzyme from Coleus forskohlii. Thus enzyme IV may be a CYP450 from Coleus forskohlii.

In one embodiment of the invention, enzyme IV is selected from the group consisting of CYP76AH11, CYP71 D381 and CYP76AH9. Thus, enzyme IV may be CYP76AH11 of SEQ ID NO:9, CYP71 D381 of SEQ ID NO:10, CYP76AH9 of SEQ ID NO:11 or a functional homologue of any of the aforementioned sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

In particular, in embodiments of the invention wherein enzyme IV is capable of catalysing reaction IVa, then enzyme IV may be CYP76AH11. Thus, enzyme IV may be CYP76AH11 in embodiments of the invention, wherein enzyme IV is capable of catalysing reaction IVa, wherein R₁ and R₅ are —H and R₂ is —OH. Thus, enzyme IV may be CYP76AH11 of SEQ ID NO:9 or any of the functional homologues thereof described herein above also capable of catalysing reaction IVa.

In embodiments of the invention wherein enzyme IV is capable of catalysing reaction IVb, then enzyme IV may in particular be CYP71 D381 or CYP76AH9. Thus, enzyme IV may be CYP71 D381 of SEQ ID NO:10 or CYP76AH9 of SEQ ID NO:11 or any of the functional homologues thereof described herein above.

The heterologous nucleic acid encoding the enzyme III may be any heterologous nucleic acid encoding any of the enzyme Ills described herein in this section. Thus, in one embodiment the heterologous nucleic acid may be any heterologous nucleic acid encoding CYP76AH11 of SEQ ID NO:9 or any of the functional homologues thereof described herein above. In one embodiment the heterologous nucleic acid may comprise or consist of the sequence described in international patent application WO2015113569 as SEQ ID NO:6.

V. Enzyme Catalysing Transfer of an Acyl Group

In addition to the heterologous nucleic acid encoding the enzyme capable of catalysing hydroxylation of 13R-MO and/or oxidised 13R-MO at the 9 position, the host organism may comprise a heterologous nucleic acid encoding an enzyme capable of catalysing transfer of an acyl group to an —OH of a hydroxylated 13R-MO and/or an oxidised hydroxylated-13R-MO.

Said enzyme may for example be any of the enzymes described herein in this section and may also be referred to herein as “enzyme V”. It is in particular preferred that the host organism comprises a heterologous nucleic acid encoding said enzyme, in embodiments of the invention, wherein the oxidised 13R-MO to be produced is substituted at one of the positions 1, 6, 7, 9 or 11 with —OR, wherein R preferably is acyl.

The enzyme V may for example be capable of catalysing the following reaction Va:

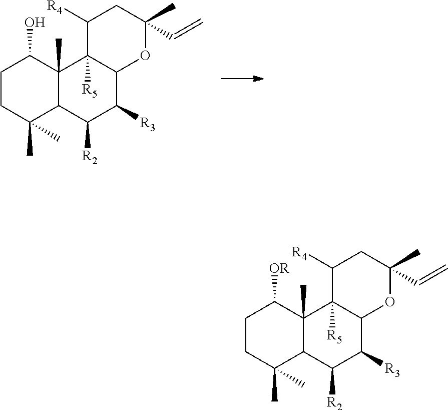

wherein R is acyl, more preferably R is acetyl and

R₂, R₃ and R₅ individually are selected from the group consisting of —H, —OH and —OX, wherein X preferably is acyl and

R₄ is —H, —OH or ═O. Acyl is as defined in the section “Oxidised 13R-MO” herein below.

In particular, at least one of R₂, R₃ and R₅ is —H or —OH, for example at least two of R₂, R₃ and R₅ is —H or —OH, for example all of R₂, R₃ and R₅ is —H or —OH.

The enzyme V may for example be capable of catalysing the following reaction Vb:

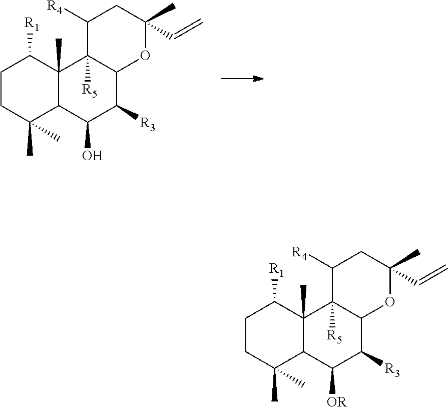

wherein R is acyl, more preferably R is acetyl and

R₁, R₃ and R₅ individually are selected from the group consisting of —H, —OH and —OX, wherein X preferably is acyl and

R₄ is —H, —OH or ═O. Acyl is as defined in the section “Oxidised 13R-MO” herein below.

In particular, at least one of R₁, R₃ and R₅ is —H or —OH, for example at least two of R₁, R₃ and R₅ is —H or —OH, for example all of R₁, R₃ and R₅ is —H or —OH.

The enzyme V may for example be capable of catalysing the following reaction Vc:

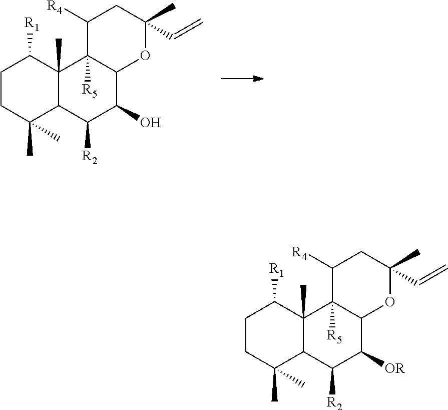

wherein R is acyl, more preferably R is acetyl and

R₁, R₂ and R₅ individually are selected from the group consisting of —H, —OH and —OX, wherein X preferably is acyl and

R₄ is —H, —OH or ═O. Acyl is as defined in the section “Oxidised 13R-MO” herein below.

In particular, at least one of R₁, R₂ and R₅ is —H or —OH, for example at least two of R₁, R₂ and R₅ is —H or —OH, for example all of R₁, R₂ and R₅ is —H or —OH.

The enzyme V may be capable of catalysing one or more of the reactions Va, Vb and Vc outlined above.

Enzyme V may be any useful enzyme with above mentioned activities, in particular enzyme V may be an acyl transferase. Enzyme V may be derived from any suitable source, but in a preferred embodiment enzyme V is an enzyme from Coleus forskohlii. Thus enzyme V may be a acyl transferase from Coleus forskohlii.

VI. TPS2

In addition to the heterologous nucleic acid encoding the enzyme capable of catalysing hydroxylation of 13R-MO and/or oxidised 13R-MO at the 9 position, the host organism may comprise a heterologous nucleic acid encoding TPS2. It is preferred that in embodiments of the invention where the host organism comprises a nucleic acid encoding TPS2, then the host organism also comprises a heterologous nucleic acid encoding either TPS3 or TPS4.

Said TPS2 may for example be any of the enzymes described herein in this section and may also be referred to herein as “enzyme VI”.

Preferably said TPS2 is an enzyme capable of catalysing the reaction VI:

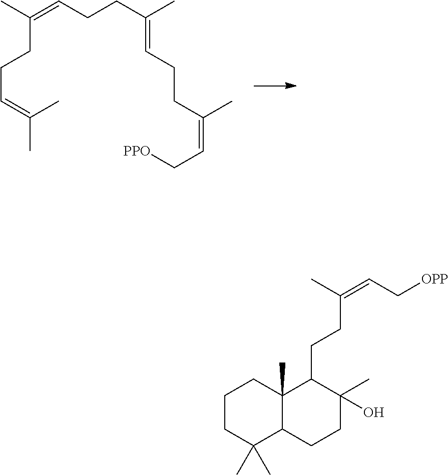

wherein —OPP refers to diphosphate.

In particular, it is preferred that said TPS2 is TPS2 of Coleus forskohlii, also known as CfTPS2. In particular, said enzyme VI may be a polypeptide of SEQ ID NO:3 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

The sequence identity is preferably calculated as described herein below in the section “Sequence identity”. A functional homologue of a TPS2 is a polypeptide, which is also capable of catalysing reaction VI described above.

VII. TPS3

In addition to the heterologous nucleic acid encoding the enzyme capable of catalysing hydroxylation of 13R-MO and/or oxidised 13R-MO at the 9 position, the host organism may comprise a heterologous nucleic acid encoding TPS3. It is preferred that in embodiments of the invention where the host organism comprises a nucleic acid encoding TPS3, then the host organism also comprises a heterologous nucleic acid encoding TPS2.

Said TPS3 may for example be any of the enzymes described herein in this section and may also be referred to herein as “enzyme VII”.

Preferably said TPS3 is an enzyme capable of catalysing the reaction VII:

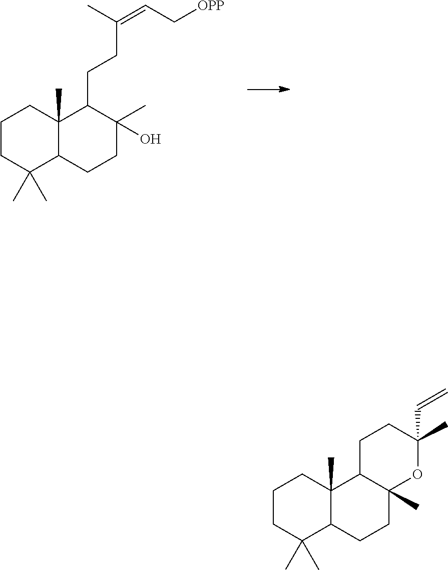

In particular, it is preferred that said TPS3 is TPS3 of Coleus forskohlii also known as CfTPS3. In particular, said enzyme VII may be a polypeptide of SEQ ID NO:4 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

The sequence identity is preferably calculated as described herein below in the section “Sequence identity”. A functional homologue of a TPS2 is a polypeptide, which is also capable of catalysing reaction VII described above.

VIII. TPS4

In addition to the heterologous nucleic acid encoding the enzyme capable of catalysing hydroxylation of 13R-MO and/or oxidised 13R-MO at the 9 position, the host organism may comprise a heterologous nucleic acid encoding TPS4. It is preferred that in embodiments of the invention where the host organism comprises a nucleic acid encoding TPS4, then the host organism also comprises a heterologous nucleic acid encoding TPS2.

Said TPS4 may for example be any of the enzymes described herein in this section and may also be referred to herein as “enzyme VIII”.

Preferably said TPS4 is an enzyme capable of catalysing the reaction VIII:

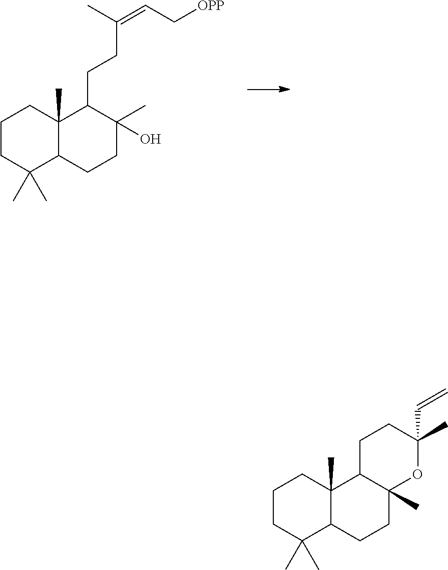

In particular, it is preferred that said TPS4 is TPS4 of Coleus forskohlii also known as CfTPS4. In particular, said enzyme VIII may be a polypeptide of SEQ ID NO:5 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

The sequence identity is preferably calculated as described herein below in the section “Sequence identity”. A functional homologue of a TPS4 is a polypeptide, which is also capable of catalysing reaction VIII described above.

IX. GGPP

The host organism may be capable of production of GGPP. For example the host organism may produce GGPP endogenously. The host organism may also be recombinantly modulated to produce GGPP. Even if the host organism produces GGPP endogenously, the host organism may be recombinantly modulated to upregulate production of GGPP. This may be achieved by expressing one or more enzymes involved in the production of GGPP in said host organism. In addition or alternatively, the expression of one or more enzymes involved in reducing the level of GGPP may be reduced or even abolished in the host organism.

Thus, the host organism may comprise one or more heterologous nucleic acids encoding enzymes involved in the synthesis of GGPP. Said enzymes may also be referred to as “enzyme IX” herein. Enzyme IX may for example be a GGPP synthase (GGPPS).

In one embodiment the host organism may comprise a heterologous nucleic acid encoding an enzyme IX, wherein said enzyme IX is a GGPP synthase (GGPPS), for example a GGPPS1, such as GGPPS1 of Coleus forskohlii, e.g. CfGGPPs. The GGPPS may be a polypeptide encoded by SEQ ID NO:6 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

It is also comprised within the invention that the host organism comprise a heterologous nucleic acid encoding a GGPPS (e.g. any of the GGPPS described above).

X. 1-deoxy-D-xylulose-5-phosphate Synthase

The host organism may in addition to one or more of the heterologous nucleic acids described herein above also comprise one or more heterologous nucleic acids encoding a 1-deoxy-D-xylulose-5-phosphate synthase, which may also be referred to as “Enzyme X” herein. Enzyme X may for example be a DXS.

In one embodiment, the host organism may comprise a heterologous nucleic encoding an enzyme X, wherein said enzyme X is a DXS, such as DXS of Coleus forskohlii, e.g. CfDXS. DXS may be a polypeptide encoded by SEQ ID NO:7 or a functional homologue thereof sharing at least 70%, such as at least 80%, for example at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99%, such as 100% sequence identity therewith.

It is also comprised within the invention that the host organism comprises a heterologous nucleic acid encoding a DXS (e.g. any of the DXS described above).

Sequence Identity

A high level of sequence identity indicates likelihood that the first sequence is derived from the second sequence. Amino acid sequence identity requires identical amino acid sequences between two aligned sequences. Thus, a candidate sequence sharing 80% amino acid identity with a reference sequence, requires that, following alignment, 80% of the amino acids in the candidate sequence are identical to the corresponding amino acids in the reference sequence. Identity according to the present invention is determined by aid of computer analysis, such as, without limitations, the ClustalW computer alignment program (Higgins D., Thompson J., Gibson T., Thompson J. D., Higgins D. G., Gibson T. J., 1994. CLUSTAL W: improving the sensitivity of progressive multiple sequence alignment through sequence weighting, position-specific gap penalties and weight matrix choice. Nucleic Acids Res. 22:4673-4680), and the default parameters suggested therein. The ClustalW software is available from as a ClustalW WWW Service at the European Bioinformatics Institute http://www.ebi.ac.uk/clustalw. Using this program with its default settings, the mature (bioactive) part of a query and a reference polypeptide are aligned. The number of fully conserved residues are counted and divided by the length of the reference polypeptide.

The ClustalW algorithm may similary be used to align nucleotide sequences. Sequence identities may be calculated in a similar way as indicated for amino acid sequences. In one important embodiment, the cell of the present invention comprises a nucleic acid sequence coding, as define herein.

Heterologous Nucleic Acid

The term “heterologous nucleic acid” as used herein refers to a nucleic acid sequence, which has been introduced into the host organism, wherein said host does not endogenously comprise said nucleic acid. For example, said heterologous nucleic acid may be introduced into the host organism by recombinant methods. Thus, the genome of the host organism has been augmented by at least one incorporated heterologous nucleic acid sequence. It will be appreciated that typically the genome of a recombinant host described herein is augmented through the stable introduction of one or more heterologous nucleic acids encoding one or more enzymes.

Suitable host organisms include microorganisms, plant cells, and plants, and may for example be any of the host organisms described herein below in the section “Host organism”.

In general the heterologous nucleic acid encoding a polypeptide (also referred to as “coding sequence” in the following) is operably linked in sense orientation to one or more regulatory regions suitable for expressing the polypeptide. Because many microorganisms are capable of expressing multiple gene products from a polycistronic mRNA, multiple polypeptides can be expressed under the control of a single regulatory region for those microorganisms, if desired. A coding sequence and a regulatory region are considered to be operably linked when the regulatory region and coding sequence are positioned so that the regulatory region is effective for regulating transcription or translation of the sequence. Typically, the translation initiation site of the translational reading frame of the coding sequence is positioned between one and about fifty nucleotides downstream of the regulatory region for a monocistronic gene.

“Regulatory region” refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also may include at least one control element, such as an enhancer sequence, an upstream element or an upstream activation region (UAR). A regulatory region is operably linked to a coding sequence by positioning the regulatory region and the coding sequence so that the regulatory region is effective for regulating transcription or translation of the sequence. For example, to operably link a coding sequence and a promoter sequence, the translation initiation site of the translational reading frame of the coding sequence is typically positioned between one and about fifty nucleotides downstream of the promoter. A regulatory region can, however, be positioned at further distance, for example as much as about 5,000 nucleotides upstream of the translation initiation site, or about 2,000 nucleotides upstream of the transcription start site.

The choice of regulatory regions to be included depends upon several factors, including the type of host organism. It is a routine matter for one of skill in the art to modulate the expression of a coding sequence by appropriately selecting and positioning regulatory regions relative to the coding sequence. It will be understood that more than one regulatory region may be present, e.g., introns, enhancers, upstream activation regions, transcription terminators, and inducible elements.

It will be appreciated that because of the degeneracy of the genetic code, a number of nucleic acids can encode a particular polypeptide; i.e., for many amino acids, there is more than one nucleotide triplet that serves as the codon for the amino acid. Thus, codons in the coding sequence for a given polypeptide can be modified such that optimal expression in a particular host organisms obtained, using appropriate codon bias tables for that host (e.g., microorganism). Nucleic acids may also be optimized to a GC-content preferable to a particular host, and/or to reduce the number of repeat sequences. As isolated nucleic acids, these modified sequences can exist as purified molecules and can be incorporated into a vector or a virus for use in constructing modules for recombinant nucleic acid constructs.

A non-limiting example of a heterologous nucleic acid encoding CYP76AH16 is provided herein as SEQ ID NO:1. Thus, the heterologous nucleic acid encoding the enzyme capable of catalysing hydroxylation of 13R-MO and/or oxidised 13R-MO at the 9 position may comprise or consist of SEQ ID NO:1 or a sequence sharing at least 70%, such as at least 80%, for example at least 90%, such as at least 95% sequence identity therewith.

Oxidised 13R-MO

The present invention relates to methods for producing forskolin and related compounds. In particular, the invention relates to methods for producing oxidised 13R-MO.

The term “oxidised 13R-MO” as used herein refers to 13R-manoyl-oxide (13R-MO) substituted at one or more positions with a moiety selected from the group consisting of ═O, —OH and OR, wherein R preferably is acyl.

The term “substituted with a moiety” as used herein in relation to chemical compounds refers to hydrogen group(s) being substituted with said moiety.

The term “acyl” as used herein denoted a substituent of the formula —(C═O)-alkyl. “Alkyl” as used herein refers to a saturated, straight or branched hydrocarbon chain. The hydrocarbon chain preferably contains of from one to eighteen carbon atoms (C₁₋₁₈-alkyl), more preferred of from one to six carbon atoms (C₁₋₆-alkyl), including methyl, ethyl, propyl, isopropyl, butyl, isobutyl, secondary butyl, tertiary butyl, pentyl, isopentyl, neopentyl, tertiary pentyl, hexyl and isohexyl. In a preferred embodiment alkyl represents a C₁₋₃-alkyl group, which may in particular include methyl, ethyl, propyl or isopropyl. In another preferred embodiment of this invention alkyl represents methyl.

The term “oxo” as used herein refers to a “═O” substituent.

The term “keto-” as used herein is used as a prefix to indicate possession of a carbonyl (C=O) group.

The term “hydroxyl” as used herein refers to a “—OH” substituent.

The structure of 13R-manoyl-oxide (13R-MO) is provided below. The structure also provides the numbering of the carbon atoms of the ring structure used herein.

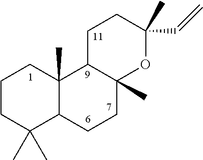 [SMILES: C=C[C@@]1(C)CCC2[C@@]3(C)CCCC(C)(C)C3CC[C@@]2(C)O1]

Preferably, the oxidised 13R-MO according to the present invention, is 13R-MO substituted at one or more of the positions 1, 6, 7, 9 and/or 11 with a moiety selected from the group consisting of ═O, —OH and OR, wherein R preferably is acyl.

In another embodiment of the invention the oxidised 13R-MO is 13R-MO substituted at the 2 position with —OH. In yet another embodiment of the invention the oxidised 13R-MO is 13R-MO substituted on the methyl at the 10 position with —OH. Thus, the substituent on the 1 position may be —CH₂—OH.

In particular the oxidised 13R-MO may be a compound of formula III:

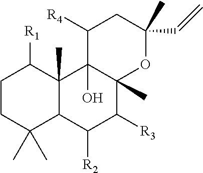

wherein R₁, R₂, R₃, and R₄ individually are selected from the group consisting of, —H ═O, —OH and —OR, wherein R preferably is acyl, more preferably R is —(C═O)—CH₃.

For example, R₁ may be selected from the group consisting of —OH, —H and ═O.

For example R₂ may be selected from the group consisting of —H, —OH and —O-acyl, for example R₂ may be selected from the group consisting of —H, —OH and —O—(C═O)—CH₃.

For example R₃ may be selected from the group consisting of —H, —OH and —O-acyl, for example R₃ may be selected from the group consisting of —H, —OH and —O—(C═O)—CH₃.

For example R₄ may be selected from the group consisting of —H, —OH, ═O and —O-acyl, for example R₅ may be selected from the group consisting of ═O and O—(C═O)—CH₃.

In particular, the oxidised 13R-MO may be 13R-MO, which is substituted at the 11 position with ═O and at the 9-postion with —OH. In addition, to said substitution, the oxidised 13R-MO may be substituted at one or more of the positions 1, 6 and 7 with a moiety selected from the group consisting of ═O, —OH and OR, preferably with a moiety selected from the group consisting of ═O, —OH and —OR. R may be acyl, wherein acyl is as defined above.

Thus, in one embodiment of the invention said oxidised 13R-MO is a compound of the formula I

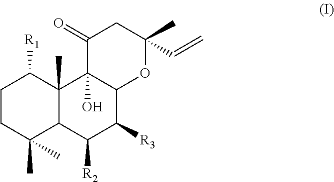

wherein R₁, R₂ and R₃ individually are selected from the group consisting of —H, —OH and —OR, wherein R preferably is acyl, wherein acyl is as defined above. It is contained within the invention that at least one of R₁, R₂ and R₃ may be —OH or —OR, for example at least two of R₁, R₂ and R₃ may be —OH or —OR, for example all of R₁, R₂ and R₃ and R₄ may be —OH or —OR.

R₁ may be selected from the group consisting of —H, —OH and —OR, wherein R is as defined above. Preferably, R₁ is selected from the group consisting of —H and —OH, in particular R₁ may be —OH.

R₂ may be selected from the group consisting of —H, —OH and —OR, wherein R is as defined above. Preferably, R₂ is selected from the group consisting of —OR and —OH, wherein R is as defined above. For example R₂ may be selected from the group consisting of —O—(C═O)-CH₃ (acetyl), —O—(C═O)—CH₂—CH₃, —O—(C═O)—CH₂—CH₂-CH₃ and —OH. In particular, R₂ may be —OH.

R₃ may be selected from the group consisting of —H, —OH and —OR, wherein R is as defined above. Preferably, R₃ is selected from the group consisting of —OR and —OH, wherein R is as defined above. For example R₃ may be selected from the group consisting of —O—(C═O)—CH₃ (acetyl), —O—(C═O)—CH₂—CH₂—CH₃ O—(C═O)—CH₂—CH₂—CH₃ and —OH. In particular, R₃ may be acetyl.

It is also comprised within the invention that the oxidised 13R-MO is not substituted at the 11 position. Thus, the oxidised 13R-MO may be a compound of the formula (II)

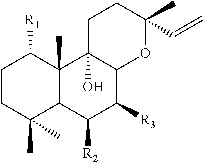

wherein each of R₁, R₂ and R₃ may be as indicated herein above in relation to compounds of formula I.

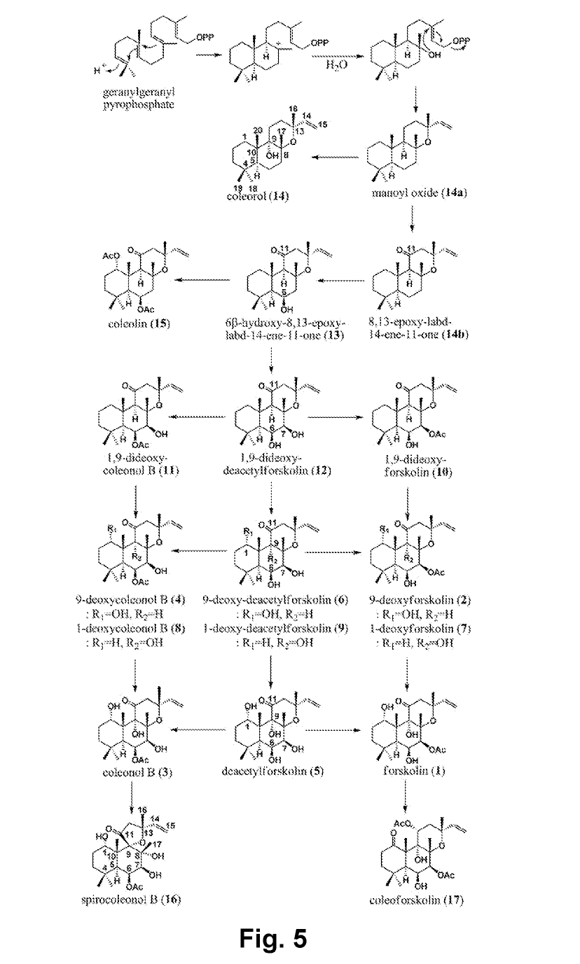

For example, the oxidised 13R-MO may be selected from the group consisting of compounds 1, 3, 5, 7, 8, 9 and 14 and shown in FIG. 5.

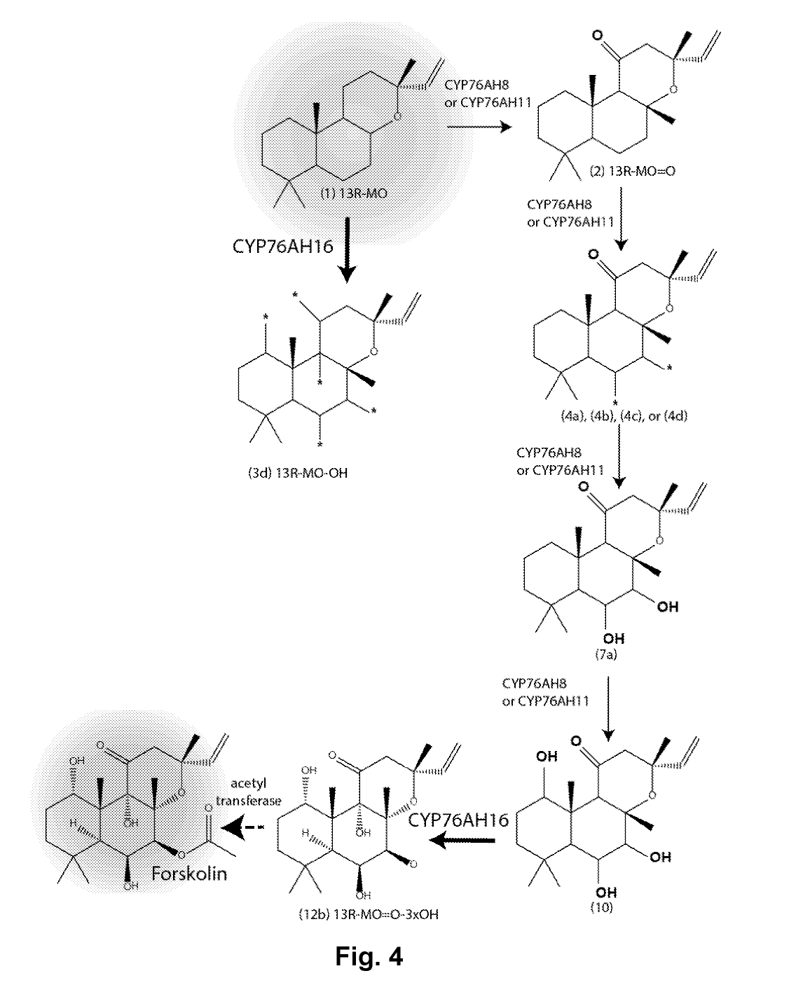

The oxidised 13R-MO may also be any of the oxidised 13R-MO shown in FIG. 4, such as compounds 2, 3d, 4a, 4b, 4c, 4d, 7a, 10 or 12b of FIG. 4.

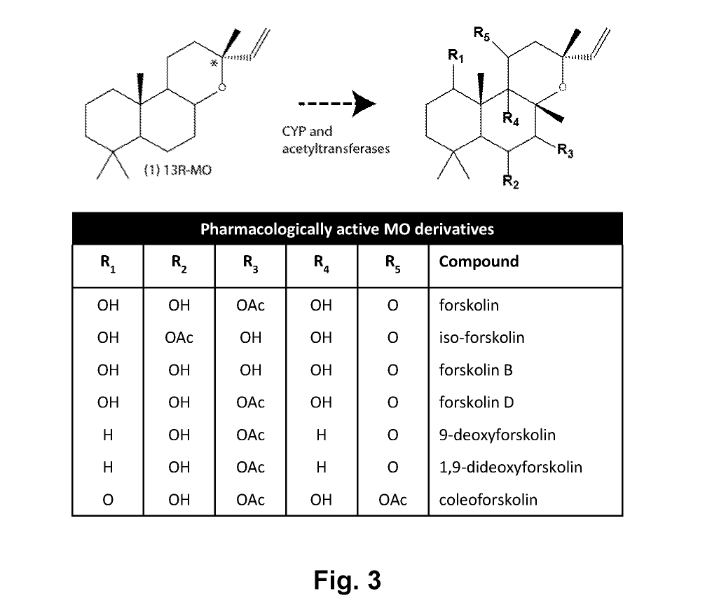

In particular, the oxidised 13R-MO may be selected from the group consisting of forskolin, iso-forskolin, forskolin B, forskolin D and coleoforskolin. The structures of these compounds are provided in FIG. 3.

In a preferred embodiment of the invention, the oxidised 13R-MO is deacetylforskolin, e.g. the oxidised 13R-MO is the compound 12b shown in FIG. 4.

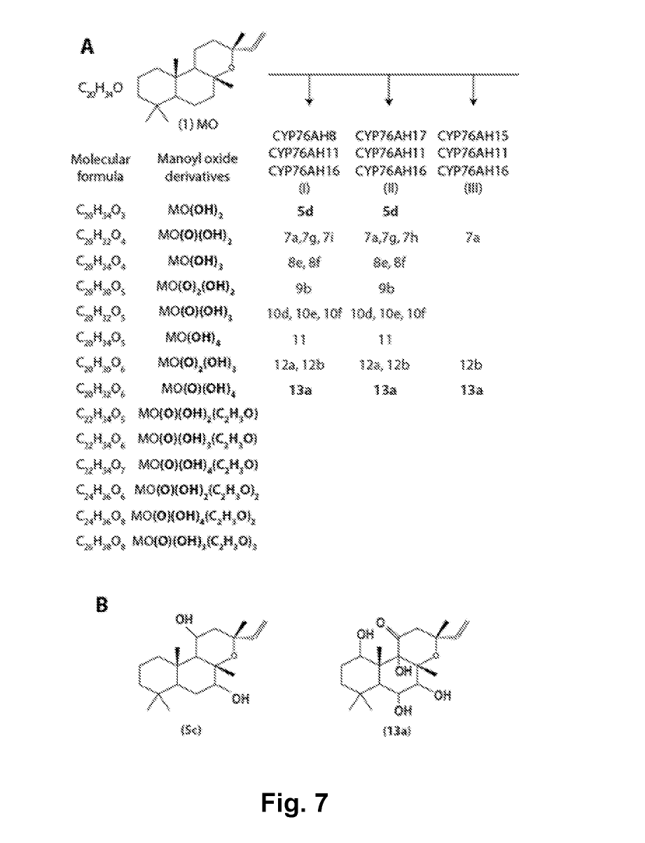

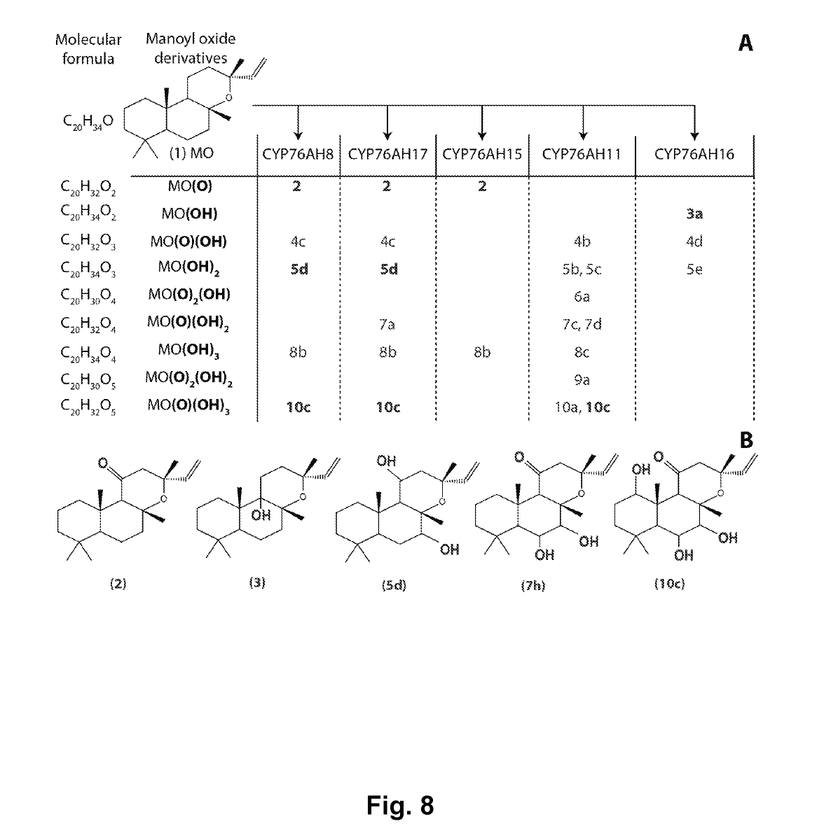

In other preferred embodiments of the invention, the oxidised 13R-MO is the compound shown as compound 3 in FIG. 8 or the compound shown as compound 13a in FIG. 7.

In a preferred embodiment of the invention, the invention relates to methods for producing forskolin. The term “forskolin” as used herein refers to a compound of the formula

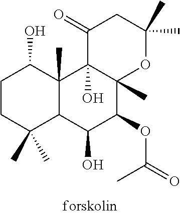

Host Organism

The host organism to be used with the methods of the invention, may be any suitable host organism containing a heterologous nucleic acid encoding an enzyme capable of catalysing hydroxylation of 13R-MO and/or oxidised 13R-MO at the 9 position. In addition the host organism may contain one or more of the heterologous nucleic acids encoding enzymes I., II., III., IV., V., VI, VII, VIII, IX and/or X. described herein above.

In a preferred embodiment of the invention the host organism comprises at least the following heterologous nucleic acids:
a) a heterologous nucleic acid encoding CYP76AH16, e.g. CYP76AH16 of SEQ ID NO:2 or a functional homologue thereof; and
b) a heterologous nucleic acid encoding TPS2, e.g. CfTPS2 of SEQ ID NO:3 or a functional homologue thereof; and
c) a heterologous nucleic acid encoding TPS3, e.g. CfTPS3 of SEQ ID NO:4 or a functional homologue thereof.

In another preferred embodiment of the invention the host organism comprises at least the following heterologous nucleic acids:
a) a heterologous nucleic acid encoding CYP76AH16, e.g. CYP76AH16 of SEQ ID NO:2 or a functional homologue thereof; and
b) a heterologous nucleic acid encoding TPS2, e.g. CfTPS2 of SEQ ID NO:3 or a functional homologue thereof; and
c) a heterologous nucleic acid encoding TPS4, e.g. CfTPS4 of SEQ ID NO:5 or a functional homologue thereof.

In another preferred embodiment of the invention the host organism comprises at least the following heterologous nucleic acids:
a) a heterologous nucleic acid encoding CYP76AH16, e.g. CYP76AH16 of SEQ ID NO:2 or a functional homologue thereof; and
b) a heterologous nucleic acid encoding CYP76AH8, e.g. CYP76AH8 from Coleus forskohlii of SEQ ID NO:8 or a functional homologue thereof; and
c) a heterologous nucleic acid encoding CYP76AH11, e.g. CYP76AH11 of SEQ ID NO:9 from Coleus forskohlii or a functional homologue thereof; and
d) a heterologous nucleic acid encoding TPS2, e.g. CfTPS2 of SEQ ID NO:3 or a functional homologue thereof; and
e) a heterologous nucleic acid encoding TPS3, e.g. CfTPS3 of SEQ ID NO:4 or a functional homologue thereof.

In another preferred embodiment of the invention the host organism comprises at least the following heterologous nucleic acids:
a) a heterologous nucleic acid encoding CYP76AH16, e.g. CYP76AH16 of SEQ ID NO:2 or a functional homologue thereof; and
b) a heterologous nucleic acid encoding CYP76AH15, e.g. CYP76AH15 of SEQ ID NO:13 or a functional homologue thereof; and
c) a heterologous nucleic acid encoding CYP76AH11, e.g. CYP76AH11 of SEQ ID NO:9 or a functional homologue thereof; and
d) a heterologous nucleic acid encoding TPS2, e.g. CfTPS2 of SEQ ID NO:3 or a functional homologue thereof; and
e) a heterologous nucleic acid encoding TPS3, e.g. CfTPS3 of SEQ ID NO:4 or a functional homologue thereof.

In another preferred embodiment of the invention the host organism comprises at least the following heterologous nucleic acids:
a) a heterologous nucleic acid encoding CYP76AH16, e.g. CYP76AH16 of SEQ ID NO:2 or a functional homologue thereof; and
b) a heterologous nucleic acid encoding CYP76AH17, e.g. CYP76AH17 of SEQ ID NO:12 or a functional homologue thereof; and
c) a heterologous nucleic acid encoding CYP76AH11, e.g. CYP76AH11 of SEQ ID NO:9 or a functional homologue thereof; and
d) a heterologous nucleic acid encoding TPS2, e.g. CfTPS2 of SEQ ID NO:3 or a functional homologue thereof; and
e) a heterologous nucleic acid encoding TPS3, e.g. CfTPS3 of SEQ ID NO:4 or a functional homologue thereof.

In another preferred embodiment of the invention the host organism comprises at least the following heterologous nucleic acids:
a) a heterologous nucleic acid encoding CYP76AH16, e.g. CYP76AH16 of SEQ ID NO:2 or a functional homologue thereof; and
b) one or more heterologous nucleic acids selected from the group consisting of heterologous nucleic acids encoding CYP76AH8, CYP76AH15 and CYP76AH17, e.g. CYP76AH8 from Coleus forskohlii of SEQ ID NO:8, CYP76AH15 of SEQ ID NO:13, CYP76AH17 of SEQ ID NO:12 or a functional homologue of any of the aforementioned; and
c) a heterologous nucleic acid encoding CYP76AH11, e.g. CYP76AH11 of SEQ ID NO:9 from Coleus forskohlii or a functional homologue thereof; and
d) a heterologous nucleic acid encoding TPS2, e.g. CfTPS2 of SEQ ID NO:3 or a functional homologue thereof; and
e) a heterologous nucleic acid encoding TPS4, e.g. CfTPS4 of SEQ ID NO:5 or a functional homologue thereof.

In another preferred embodiment of the invention the host organism comprises at least the following heterologous nucleic acids:
a) a heterologous nucleic acid encoding CYP76AH16, e.g. CYP76AH16 of SEQ ID NO:2 or a functional homologue thereof; and
b) a heterologous nucleic acid encoding CYP76AH8, e.g. CYP76AH8 from Coleus forskohlii of SEQ ID NO:8 or a functional homologue thereof; and
c) a heterologous nucleic acid encoding CYP76AH11, e.g. CYP76AH11 of SEQ ID NO:9 from Coleus forskohlii or a functional homologue thereof; and
d) a heterologous nucleic acid encoding TPS2, e.g. CfTPS2 of SEQ ID NO:3 or a functional homologue thereof; and
e) a heterologous nucleic acid encoding TPS3, e.g. CfTPS3 of SEQ ID NO:4 or a functional homologue thereof; and
f) a heterologous nucleic acid encoding a GGPP synthase, such as CfGGPPS of SEQ ID NO:6 or a functional homologue thereof; and
g) a heterologous nucleic acid encoding a DXS, such as Cf DXS of SEQ ID NO:7 or a functional homologue thereof.

In another preferred embodiment of the invention the host organism comprises at least the following heterologous nucleic acids:
a) a heterologous nucleic acid encoding CYP76AH16, e.g. CYP76AH16 of SEQ ID NO:2 or a functional homologue thereof; and
b) a heterologous nucleic acid encoding CYP76AH15, e.g. CYP76AH15 of SEQ ID NO:13 or a functional homologue thereof; and
c) a heterologous nucleic acid encoding CYP76AH11, e.g. CYP76AH11 of SEQ ID NO:9 or a functional homologue thereof; and
d) a heterologous nucleic acid encoding TPS2, e.g. CfTPS2 of SEQ ID NO:3 or a functional homologue thereof; and
e) a heterologous nucleic acid encoding TPS3, e.g. CfTPS3 of SEQ ID NO:4 or a functional homologue thereof.
f) a heterologous nucleic acid encoding a GGPP synthase, such as CfGGPPS of SEQ ID NO:6 or a functional homologue thereof; and
g) a heterologous nucleic acid encoding a DXS, such as Cf DXS of SEQ ID NO:7 or a functional homologue thereof.

In another preferred embodiment of the invention the host organism comprises at least the following heterologous nucleic acids:
a) a heterologous nucleic acid encoding CYP76AH16, e.g. CYP76AH16 of SEQ ID NO:2 or a functional homologue thereof; and
b) a heterologous nucleic acid encoding CYP76AH17, e.g. CYP76AH17 of SEQ ID NO:12 or a functional homologue thereof; and
c) a heterologous nucleic acid encoding CYP76AH11, e.g. CYP76AH11 of SEQ ID NO:9 or a functional homologue thereof; and
d) a heterologous nucleic acid encoding TPS2, e.g. CfTPS2 of SEQ ID NO:3 or a functional homologue thereof; and
h) a heterologous nucleic acid encoding TPS3, e.g. CfTPS3 of SEQ ID NO:4 or a functional homologue thereof.
i) a heterologous nucleic acid encoding a GGPP synthase, such as CfGGPPS of SEQ ID NO:6 or a functional homologue thereof; and
j) a heterologous nucleic acid encoding a DXS, such as Cf DXS of SEQ ID NO:7 or a functional homologue thereof.

Useful functional homologues of CYP76AH16 of SEQ ID NO:2, CfTPS2 of SEQ ID NO:3, CfTPS3 of SEQ ID NO:4, CfTPS4 of SEQ ID NO:5, CfGGPPS of SEQ ID NO:6, Cf DXS of SEQ ID NO:7, CYP76AH8 of SEQ ID NO:8, CYP76AH11 of SEQ ID NO:9, CYP76AH17 of SEQ ID NO:12 and CYP76AH15 of SEQ ID NO:13 are described herein above.

Suitable host organisms include microorganisms, plant cells, and plants.

The microorganism can be any microorganism suitable for expression of heterologous nucleic acids. In one embodiment the host organism of the invention is a eukaryotic cell. In another embodiment the host organism is a prokaryotic cell. In a preferred embodiment, the host organism is a fungal cell such as a yeast or filamentous fungus. In particular the host organism may be a yeast cell.

In a further embodiment the yeast cell is selected from the group consisting of Saccharomyces cerevisiae, Schizosaccharomyces pombe, Yarrowia lipolytica, Candida glabrata, Ashbya gossypii, Cyberlindnera jadinii, and Candida albicans.

In general, yeasts and fungi are excellent microorganism to be used with the present invention. They offer a desired ease of genetic manipulation and rapid growth to high cell densities on inexpensive media. For instance yeasts grow on a wide range of carbon sources and are not restricted to glucose. Thus, the microorganism to be used with the present invention may be selected from the group of yeasts described below:

Arxula adeninivorans (Blastobotrys adeninivorans) is a dimorphic yeast (it grows as a budding yeast like the baker's yeast up to a temperature of 42 ° C., above this threshold it grows in a filamentous form) with unusual biochemical characteristics. It can grow on a wide range of substrates and can assimilate nitrate. It has successfully been applied to the generation of strains that can produce natural plastics or the development of a biosensor for estrogens in environmental samples.

Candida boidinii is a methylotrophic yeast (it can grow on methanol). Like other methylotrophic species such as Hansenula polymorpha and Pichia pastoris, it provides an excellent platform for the production of heterologous proteins. Yields in a multigram range of a secreted foreign protein have been reported. A computational method, IPRO, recently predicted mutations that experimentally switched the cofactor specificity of Candida boidinii xylose reductase from NADPH to NADH. Details on how to download the software implemented in Python and experimental testing of predictions are outlined in the following paper.

Hansenula polymorpha (Pichia angusta) is another methylotrophic yeast (see Candida boidinii). It can furthermore grow on a wide range of other substrates; it is thermo-tolerant and can assimilate nitrate (see also Kluyveromyces lactis). It has been applied to the production of hepatitis B vaccines, insulin and interferon alpha-2a for the treatment of hepatitis C, furthermore to a range of technical enzymes.

Kluyveromyces lactis is a yeast regularly applied to the production of kefir. It can grow on several sugars, most importantly on lactose which is present in milk and whey. It has successfully been applied among others to the production of chymosin (an enzyme that is usually present in the stomach of calves) for the production of cheese. Production takes place in fermenters on a 40,000 L scale.

Pichia pastoris is a methylotrophic yeast (see Candida boidinii and Hansenula polymorpha). It provides an efficient platform for the production of foreign proteins. Platform elements are available as a kit and it is worldwide used in academia for the production of proteins. Strains have been engineered that can produce complex human N-glycan (yeast glycans are similar but not identical to those found in humans).

Saccharomyces cerevisiae is the traditional baker's yeast known for its use in brewing and baking and for the production of alcohol. As protein factory it has successfully been applied to the production of technical enzymes and of pharmaceuticals like insulin and hepatitis B vaccines. Also it has been useful for production of terpenoids.

Yarrowia lipolytica is a dimorphic yeast (see Arxula adeninivorans) that can grow on a wide range of substrates. It has a high potential for industrial applications but there are no recombinant products commercially available yet.

In another embodiment the host organism is a microalgae such as Chlorella and Prototheca.

In another embodiment of the invention the host organism is a filamentous fungus, for example Aspergillus.

In further yet another embodiment the host organism is a plant cell. The host organism may be a cell of a higher plant, but the host organism may also be cells from organisms not belonging to higher plants for example cells from the moss Physcomitrella patens.

In another embodiment the host organism is a mammalian cell, such as a human, feline, porcine, simian, canine, murine, rat, mouse or rabbit cell.

As mentioned, the host organism can also be a prokaryotic cell such as a bacterial cell. If the host organism is a prokaryotic cell the cell may be selected from, but not limited to E. coli, Corynebacterium, Bacillus, Pseudomonas and Streptomyces cells.

The host organism may also be a plant.

A plant or plant cell can be transformed by having a heterologous nucleic acid integrated into its genome, i.e., it can be stably transformed. Stably transformed cells typically retain the introduced nucleic acid with each cell division. A plant or plant cell can also be transiently transformed such that the recombinant gene is not integrated into its genome. Transiently transformed cells typically lose all or some portion of the introduced nucleic acid with each cell division such that the introduced nucleic acid cannot be detected in daughter cells after a certain number of cell divisions. Both transiently transformed and stably transformed transgenic plants and plant cells can be useful in the methods described herein.

Plant cells comprising a heterologous nucleic acid used in methods described herein can constitute part or all of a whole plant. Such plants can be grown in a manner suitable for the species under consideration, either in a growth chamber, a greenhouse, or in a field. Plants may also be progeny of an initial plant comprising a heterologous nucleic acid provided the progeny inherits the heterologous nucleic acid. Seeds produced by a transgenic plant can be grown and then selfed (or outcrossed and selfed) to obtain seeds homozygous for the nucleic acid construct.

The plants to be used with the invention can be grown in suspension culture, or tissue or organ culture. For the purposes of this invention, solid and/or liquid tissue culture techniques can be used. When using solid medium, plant cells can be placed directly onto the medium or can be placed onto a filter that is then placed in contact with the medium. When using liquid medium, transgenic plant cells can be placed onto a flotation device, e.g., a porous membrane that contacts the liquid medium.

When transiently transformed plant cells are used, a reporter sequence encoding a reporter polypeptide having a reporter activity can be included in the transformation procedure and an assay for reporter activity or expression can be performed at a suitable time after transformation. A suitable time for conducting the assay typically is about 1-21 days after transformation, e.g., about 1-14 days, about 1-7 days, or about 1-3 days. The use of transient assays is particularly convenient for rapid analysis in different species, or to confirm expression of a heterologous polypeptide whose expression has not previously been confirmed in particular recipient cells.

Techniques for introducing nucleic acids into monocotyledonous and dicotyledonous plants are known in the art, and include, without limitation, Agrobacterium-mediated transformation, viral vector-mediated transformation, electroporation and particle gun transformation, U.S. Pat. Nos. 5,538,880; 5,204,253; 6,329,571; and 6,013,863. If a cell or cultured tissue is used as the recipient tissue for transformation, plants can be regenerated from transformed cultures if desired, by techniques known to those skilled in the art.

The plant comprising a heterologous nucleic acid to be used with the present invention may for example be selected from: corn (Zea. mays), canola (Brassica napus, Brassica rapa ssp.), alfalfa (Medicago sativa), rice (Oryza sativa), rye (Secale cerale), sorghum (Sorghum bicolor, Sorghum vulgare), sunflower (Helianthus annuas), wheat (Tritium aestivum and other species), Triticale, Rye (Secale) soybean (Glycine max), tobacco (Nicotiana tabacum), potato (Solanum tuberosum), peanuts (Arachis hypogaea), cotton (Gossypium hirsutum), sweet potato (Impomoea batatus), cassava (Manihot esculenta), coffee (Cofea spp.), coconut (Cocos nucifera), pineapple (Anana comosus), citrus (Citrus spp.) cocoa (Theobroma cacao), tea (Camellia senensis), banana (Musa spp.), avacado (Persea americana), fig (Ficus casica), guava (Psidium guajava), mango (Mangifer indica), olive (Olea europaea), papaya (Carica papaya), cashew (Anacardium occidentale), macadamia (Macadamia intergrifolia), almond (Primus amygdalus), apple (Malus spp), Pear (Pyrus spp), plum and cherry tree (Prunus spp), Ribes (currant etc.), Vitis, Jerusalem artichoke (Helianthemum spp), non-cereal grasses (Grass family), sugar and fodder beets (Beta vulgaris), chicory, oats, barley, vegetables, and ornamentals.

For example, plants of the present invention are crop plants (for example, cereals and pulses, maize, wheat, potatoes, tapioca, rice, sorghum, millet, cassava, barley, pea, sugar beets, sugar cane, soybean, oilseed rape, sunflower and other root, tuber or seed crops. Other important plants maybe fruit trees, crop trees, forest trees or plants grown for their use as spices or pharmaceutical products (Mentha spp, clove, Artemesia spp, Thymus spp, Lavendula spp, Allium spp., Hypericum, Catharanthus spp, Vinca spp, Papaver spp., Digitalis spp, Rawolfia spp., Vanilla spp., Petrusilium spp., Eucalyptus, tea tree, Picea spp, Pinus spp, Abies spp, Juniperus spp,. Horticultural plants which may be used with the present invention may include lettuce, endive, and vegetable brassicas including cabbage, broccoli, and cauliflower, carrots, and carnations and geraniums.

The plant may also be selected from the group consisting of tobacco, cucurbits, carrot, strawberry, sunflower, tomato, pepper and Chrysanthemum.

The plant may also be a grain plants for example oil-seed plants or leguminous plants. Seeds of interest include grain seeds, such as corn, wheat, barley, sorghum, rye, etc. Oil-seed plants include cotton soybean, safflower, sunflower, Brassica, maize, alfalfa, palm, coconut, etc. Leguminous plants include beans and peas. Beans include guar, locust bean, fenugreek, soybean, garden beans, cowpea, mung bean, lima bean, fava bean, lentils, chickpea.

In a further embodiment of the invention said plant is selected from the following group: maize, rice, wheat, sugar beet, sugar cane, tobacco, oil seed rape, potato and soybean. Thus, the plant may for example be rice.

In one embodiment the plant is tobacco.

The whole genome of Arabidopsis thaliana plant has been sequenced (The Arabidopsis Genome Initiative (2000). “Analysis of the genome sequence of the flowering plant Arabidopsis thaliana”. Nature 408 (6814): 796-815. doi:10.1038/35048692. PMD 11130711). Consequently, very detailed knowledge is available for this plant and it may therefore be a useful plant to work with.

Accordingly, one plant, which may be used with the present invention is an Arabidopsis and in particular an Arabidopsis thaliana.

It may be preferred that the plant is not Coleus forskohlii.

Sequences

is the cDNA sequence from Coleus forskohlii
encoding CYP76AH16:
SEQ ID NO: 1
ATGGAGTTGGTGGAAGTGATTGTGGTGGTGGTGGGAGCGGCCGCGTTGGG
CGTTGTTTTGTGGTCTCACTTGAAGCCGGAGGGCAGAAAGCTTCCACCAG
GGCCTTCCCCTCTGCCTATCTTCGGAAACATTTTCCAGCTGACGGGTCCA
AACACTTGTGAATCATTTGCGAACCTCTCCAAGAAATACGGCCCCGTCAT
GTCCTTACGCCTCGGTAGCTTATTCACCGTCGTCATTTCGTCGCCGGAAA
TGGCGAAAGAAGTACTGACTAACACAGACTTTTTAGAGAGGCCCCTGATG
CAGGCGGTCCATGCGCATGACCACGCCCAGTTCTCCATTGCGTTCCTGCC
GGTGACCACCCCTAAATGGAAACAACTGCGCCGGATTTGCCAGGAGCAAA
TGTTTGCGAGTCGGATCTTGGAGAAGAGCCAGCCCCTCCGTCACCAGAAG
CTGCAGGAGCTGATCGACCACGTGCAGAAATGCTGCGACGCTGGGCGAGC
GGTTACTATCCGCGACGCCGCCTTCGCGACCACGCTCAACCTCATGTCGG
TGACGATGTTCTCGGCTGACGCTACTGAGTTGGATTCCAGCGTCACCGCG
GAGTTGAGGGAGCTCATGGCGGGCGTCGTTACTGTTCTTGGCACTCCGAA
TTTCGCCGACTTCTTCCCCATCCTCAAATATTTGGATCCGCAAGGGGTGA
GGCGCAAGGCACATTTCCACTATGGGAAGATGTTTGACCACATCAAGAGT
CGGATGGCCGAGAGAGTCGAGTTGAAAAAGGCAAACCCAAATCATCTCAA
ACACGACGACTTCTTGGAGAAAATCTTGGACATCAGTCTACGGAGGGACT
ACGAGTTGACCATCCAGGACATTACGCATTTACTGGTGGACTTGTATGTA
GCAGGAAGCGAATCAACGGTAATGTCAATTGAATGGATAATGTCTGAATT
GATGCTGCACCCGCAGTCACTAGCGAAGCTGAAAGCCGAGCTGAGAAGCG
TGATGGGAGAGAGAAAGATGATCCAGGAATCAGAGGACATATCAAGGCTT
CCCTTTCTGAATGCCGTCATCAAAGAAACCCTCCGTCTCCACCCGCCAGG
TCCCCTCCTGTTTCCCCGCCAAAACACCAACGATGTGGAACTCAATGGCT
ATTTCATCCCAAAAGGTACTCAGATACTTGTTAACGAATGGGCAATAGGC
AGAGATCCCAGTGTTTGGCCCAATCCAGAATCTTTTGTGCCGGAACGCTT
CTTGGACAAGAACATTGATTACAAAGGCCAAGATCCCCAGCTCGTCCCAT
TTGGATCGGGTAGAAGGATCTGTCTTGGGATACCCATAGCGCATCGGATG
GTGCATTCTACAGTGGCCGCATTAATTCACAATTTCGAGTGGAAGTTCGC
CCCAGACGCCACTCAATATAACACCCACTTCTTTACCCCCCCACCCCTCC
CCACCCAACTTCCTCTCAACCTCATCCCACTCAACCCTTCATTTTGA
is the protein sequence of CYP76AH16 from
Coleus forskohlii:
SEQ ID NO: 2
MELVEVIVVVVGAAALGVVLWSHLKPEGRKLPPGPSPLPIFGNIFQLTGP
NTCESFANLSKKYGPVMSLRLGSLFTVVISSPEMAKEVLTNTDFLERPLM
QAVHAHDHAQFSIAFLPVTTPKWKQLRRICQEQMFASRILEKSQPLRHQK
LQELIDHVQKCCDAGRAVTIRDAAFATTLNLMSVTMFSADATELDSSVTA
ELRELMAGVVTVLGTPNFADFFPILKYLDPQGVRRKAHFHYGKMFDHIKS
RMAERVELKKANPNHLKHDDFLEKILDISLRRDYELTIQDITHLLVDLYV
AGSESTVMSIEWIMSELMLHPQSLAKLKAELRSVMGERKMIQESEDISRL
PFLNAVIKETLRLHPPGPLLFPRQNTNDVELNGYFIPKGTQILVNEWAIG
RDPSVWPNPESFVPERFLDKNIDYKGQDPQLVPFGSGRRICLGIPIAHRM
VHSTVAALIHNFEWKFAPDCSEYNRELFSCPALRREVPLNLIPLNPSF

SEQ ID NO:3 is the protein sequence of TPS2 from Coleus forskohlii, which is described in Pateraki et al., 2014, Plant Physiology, March 2014, Vol. 164, pp. 1222-1236. The sequence has the GenBank accession number KF444507.

SEQ ID NO:4 is the protein sequence of TPS3 from Coleus forskohlii, which is described in Pateraki et al., 2014, Plant Physiology, March 2014, Vol. 164, pp. 1222-1236. The sequence has the GenBank accession number KF444508.

SEQ ID NO:5 is the protein sequence of TPS4 from Coleus forskohlii, which is described in Pateraki et al., 2014, Plant Physiology, March 2014, Vol. 164, pp. 1222-1236. The sequence has the GenBank accession number KF444509.

SEQ ID NO:6 is the protein sequence of CfGGPPs from Coleus forskohlii. The sequence has the GenBank accession number ALE19959.

SEQ ID NO:7 is the protein sequence of CfDXS from Coleus forskohlii. The sequence has the GenBank accession number ALE19960.

-amino acid sequence of CfCYP76AH8
SEQ ID NO: 8
METITLLLALFFIALTYFISSRRRRNLPPGPFPLPIIGNMLQLGSKPHQS
FAQLSKKYGPLMSIHLGSLYTVIVSSPEMAKEILQKHGQVFSGRTIAQAV
HACDHDKISMCFLPVANTWRDMRKICKEQMFSHHSLEASEELRHQKLQQL
LDYAQKCCEAGRAVDIREASFTTTLNLMSATMFSTQATEFDSEATKEFKE
TTEGVATTVGVANFADYFPILKPFDLQGIKRRADGYFGRLLKLIEGYLNE
RLESRRLNPDAPRKKDFLETLVDIIEANEYKLTTEHLTHLMLDLFVGGSE
TNTTSLEWIMSELVINPDKMAKVKEELKSVVGDEKLVNESDMPRLPYLQA
VIKEVLRIHDPGPLLLPRKAESDQVVNGYLIPKGTQILFNAWAMGRDDTI
WKDPESFEPERFLNQSIDFKGQDEELIPFGSGRRICPGMPLANRILHMTT
ATLVHNFDWKLEEGTADADHKGELFGLAVRRATPLRITPLKP
-amino acid sequence of CfCYP76AH11
SEQ ID NO: 9
MELVQVIAVVAVVVVLWSQLKRKGRKLPPGPSPLPIVGNIFQLSGKNINE
SFAKLSKIYGPVMSLRLGSLLTVIISSPEMAKEVLTSKDFANRPLTEAAH
AHGHSKFSVGFVPVSDPKWKQMRRVCQEEMFASRILENSQQRRHQKLQEL
IDHVQESRDACRAVTIRDPVFATTLNIMSLTLFSADATEFSSSATAELRD
IMACVVSVLGAANLADFFPILKYFDPQGMRRKADLHYGRLIDHIKSRMDK
RSELKKANPNHPKHDDFLEKIIDITIQRNYDLTINEITHLLVDLYLAGSE
STVMTIEWTMAELMLRPESLAKLKAELRSVMGERKMIQESDDISRLPYLN
GAIKEALRLHPPGPLLFARKSEIDVELSGYFIPKGTQILVNEWGMGRDPS
VWPNPECFQPERFLDKNIDYKGQDDQLIPFGAGRRICPGIPIAHRVVHSV
VAALVHNFDWEFAPGGSQCNNEFFTGAALVREVPLKLIPLNPPST
-amino acid sequence of CfCYP71D381
SEQ ID NO: 10
MEFDFPSALIFPAVSLLLLLWLTKTRKPKSDLDRIPGPRRLPLIGNLHHL
ISLTPPPRLFREMAAKYGPLMRLQLGGVPFLIVSSVDVAKHVVKTNDVPF
ANRPPMHAARAITYNYTDIGFAPYGEYWRNLRKICTLELLSARRVRSFRH
IREEENAGVAKWIASKEGSPANLSERVYLSSFDITSRASIGKATEEKQTL
TSSIKDAMKLGCFNVADLYPSSKLLLLITGLNFRIQRVFRKTDRILDDLL
SQHRSTSATTERPEDLVDVLLKYQKEETEVHLNNDKIKAVIMDMFLAGGE
TSATAVDWAMAEMIRNPTTLKKAQEEVRRVFDGKGYVDEEEFHELKYLKL
VIKEMLRMHPPLPFLVPRMNSERCEINGYEIPANTRLLINAWAIGRPKYW
NDAEKFIPERFENSSIDFKGNNLEYIPFGAGRRMCPGMTFGLASVEFTLA
MLLYHFDWKMPQGIKLDMTESFGASLKRKHDLLMIPTLKRPLRLAP
-amino acid sequence of CfCYP76AH9
SEQ ID NO: 11
MDFFTLLAALFLITLTFFLFFKSESKRRGGANLPPGPYPLPIVGNIFQLG
KKPHQSLAQLAKIHGPLMSLHFGSVYTVIVTSPEMAKEIFVKNDQAFLNR
TVVEAVHAHDHDKISMAFMDVGTEWRTLRRICKEQMFSTQSLETSQGLRQ
EKLQQLHDFVQRCCDSGRVVDIREASFVTTLNLMSATLFSIQATEFDSNA
TEEFREIMEGVASIVGDPNFADYFPILKRFDPQGVKRKAELYFGKMLVLV
EDLLQKRQEERRRSPSYAKKDDLLERLVDVLNEKNEYKLTTKHITHLLLD
LFVGGSETTTTSVEWIMSELLINPEKLAKLKEELKTVVGEKKQVQESDIP
QLPYFEAVLKEVFRLHPPGPLLLPRKAECDVQVGSYTIPKETQILVNAWA
IGRDPAIWPNPEAFEPERFLSQKMDYKGQDFELIPFGSGRRICPGLSFAN
RMLPMTVATLIHNFDWKLEVEANAEDVHKGEMFGIAVRRAVPLRAYPIQP
-amino acid sequence of CfCYP76AH17 (aa):
SEQ ID NO: 12
MESMNALVVGLLLIALTILFSLRRRRNLAPGPYPFPIIGNMLQLGTKPHQ
SFAQLSKKYGPLMSIHLGSLYTVIVSSPEMAKEILQKHGQVFSGRTIAQA
VHACDHDKISMGFLPVSNTWRDMRKICKEQMFSHHSLEGSQGLRQQKLLQ
LLDYAQKCCETGRAVDIREASFITTLNLMSATMGSTQATEFESKSTQEFK
EIIEGVATIVGVANFGDYFPILKPFDLQGIKRKADGYFGRLLKLIEGYLN
ERLESRKSNPNAPRKNDFLETVVDILEANEYKLSVDHLTHLMLDLFVGGS
ETNTTSLEWTMSELVNNPDKMAKLKQELKSVVGERKLVDESEMPRLPYLQ
AVIKESLRIHPPGPLLLPRKAETDQEVNGYLIPKGTQILFNVWAMGRDPS
IWKDPESFEPERFLNQNIDFKGQDFELIPFGSGRRICPCMPLANRILHMA
TATMVHNFDWKLEQGTDEADAKGELFGLAVRRAVPLRIIPLQP
amino acid sequence of CYP76AH15:
SEQ ID NO: 13
METMTLLLPLFFIALTYFLSWRRRRNLPPGPFPLPIIGNLLQIGSKPHQS
FAQLSKKYGPLMSVQLGSVYTVIASSPEMAKEILQKHGQVFSGRTIAQAA
QACGHDQISIGFLPVATTWRDMRKICKEQMFSHHSLESSKELRHEKLQKL
LDYAQKCCEAGRAVDIREAAFITTLNLMSATLFSTQATEFDSEATKEEKE
VIEGVAVIVGEPNFADYFPILKPFDLQGIKRRANSYFGRLLKLMERYLNE
RLESRRLNPDAPKKNDFLETLVDIIQADEYKLTTDHVTHLMLDLFVGGSE
TSATSLEWIMSELVSNPSKLAKVKAELKSVVGEKKVVSESEMARLDYLQA
VIKEVLRLHPPGPLLLPRKAGSDQVVNGYLIPKGTQLLFNVWAMGRDPSI
WKNPESFEPERFLNQNIDYKGQDFELIPFGSGRRICPGMPLADRIMHMTT
ATLVHNFDWKLEDGAGDADHKGDDPFGLAIRRATPLRIIPLKP

| | |
|---|---|
| SEQ ID | cDNA sequence of CYP76AH16 from Coleus forskohlii |
| NO: 1 | |
| SEQ ID | Amino acid sequence of CYP76AH16 from Coleus forskohlii |
| NO: 2 | |
| SEQ ID | Amino acid sequence of TPS2 from Coleus forskohlii (CfTPS2) |
| NO: 3 | |
| SEQ ID | Amino acid sequence of TPS3 from Coleus forskohlii (CfTPS3) |
| NO: 4 | |
| SEQ ID | Amino acid sequence of TPS4 from Coleus forskohlii (CfTPS4) |
| NO: 5 | |
| SEQ ID | Amino acid sequence of CfGGPPS from Coleus forskohlii. |
| NO: 6 | |
| SEQ ID | Amino acid sequence of CfDXS from Coleus forskohlii. |
| NO: 7 | |
| SEQ ID | Amino acid sequence of CfCYP76AH8 from Coleus forskohlii |
| NO: 8 | |
| SEQ ID | Amino acid sequence of CfCYP76AH11 from Coleus forskohlii |
| NO: 9 | |
| SEQ ID | Amino acid sequence of CfCYP71D381 from Coleus forskohlii |
| NO: 10 | |
| SEQ ID | Amino acid sequence of CfCYP76AH9 from Coleus forskohlii |
| NO: 11 | |
| SEQ ID | Amino acid sequence of CfCYP76AH17 from Coleus forskohlii |
| NO: 12 | |
| SEQ ID | Amino acid sequence of CYP76AH15 from Coleus forskohlii |
| NO: 13 | |

### EXAMPLES

The invention is further illustrated by the following examples, which however are not intended as being limiting for the invention.

### Example 1

In order to demonstrate the activity of CYP76AH16 a transient expression system in Nicotiana benthamiana was employed. The transient expression system has been described in Pateraki et al., 2014, Plant Physiology_, March 2014, Vol. 164, pp. 1222-1236, and the expression was performed essentially as described therein.

Nucleic acids encoding the following enzymes were transiently expressed:
1) CYP76AH16 of Coleus forskohlii (SEQ ID NO:2)(sequence of nucleic acid is provided as SEQ ID NO:1)
2) CYP76AH8 of Coleus forskohlii
3) CYP76AH11 of Coleus forskohlii
4) TPS2 of Coleus forskohlii (SEQ ID NO:3)
5) TPS3 of Coleus forskohlii (SEQ ID NO:4)

In addition to the above-mentioned nucleic acids in some experiments nucleic acids encoding the following enzymes were also transiently expressed:
6) CfGGPPS of Coleus forskohlii (SEQ ID NO:6)
7) Cf DXS of Coleus forskohlii (SEQ ID NO:7)

Extracts of the leaves of the Nicotiana benthamiana plants were analysed by gas-chromatography mass-spectrometry analyses as follows:

Deacetylforskolin was extracted from N. benthamiana leaf by 85% Met0H+ 15%H20+ 0.1% formic acid. The extract was subsequently analyzed by HPLC-electrospray ionisation-high resolution mass spectrometry. Separation was carried out on an Agilent 1100 SeriesHPLCunit with a Phenomenex 00F-4453-B0 (150×2 mm, Gemini NX, 3u, C18, 110A) column. The mobile phase consisted of water with 0.1% formic acid (v/v; solvent A) and acetonitrile with 0.1% formic acid (v/v; solvent B). The gradient program was 30% to 100% B over 35 min and 100% B for 1 min, followed by a return to starting conditions over 0.25 min. Chromatography (LC) unit was coupled to a Bruker microTOF mass spectrometer for accurate mass measurements.

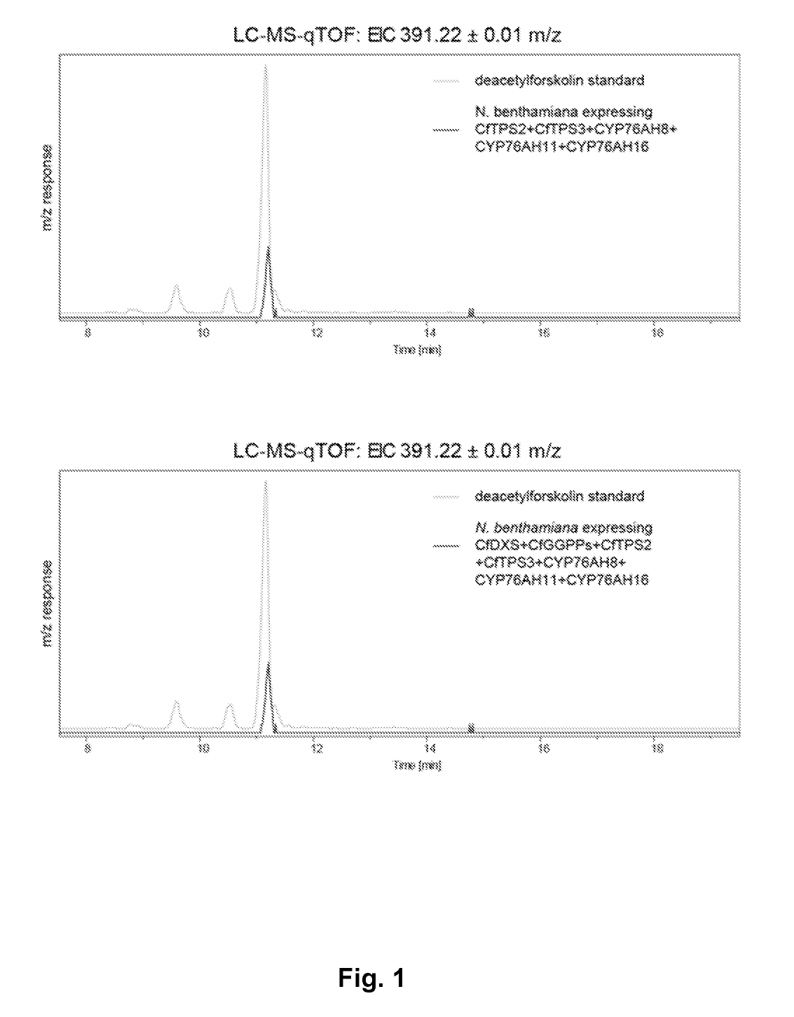

The result is shown in FIG. 1, which demonstrates the presence of deacetylforskolin. Thus, the compound extracted from N. benthamiana leaves expressing CfTPS2, CfTPS3, Cyp76AH8, CYP76AH11 a d CYP76AH16 co-eluates with and has the same molecular weight as deacetylforskolin used as standard. Similarly, the compound extracted from N. benthamiana leaves expressing CfGGPPS, CfDXS, CfTPS2, CfTPS3, CYP76AH8, CYP76AH11 and CYP76AH16 co-eluates with and has the same molecular weight as deacetylforskolin used as standard. Similar results are also observed if CYP76AH8 is exchanged with either CYP76AH15 or CYP76AH17. Thus, N. benthamiana leaves transiently expressing CfGGPPS (SEQ ID NO:6), CfDXS (SEQ ID NO:7), CfTPS2 (SEQ ID NO:3), CfTPS3 (SEQ ID NO:4), CYP76AH15 (SEQ ID NO:13), CYP76AH11 (SEQ ID NO:9) and CYP76AH16 (SEQ ID NO:2) also produces a compound co-eluating with deacetylforskolin. N. benthamiana leaves transiently expressing CfGGPPS (SEQ ID NO:6), CfDXS (SEQ ID NO:7), CfTPS2 (SEQ ID NO:3), CfTPS3 (SEQ ID NO:4), CYP76AH17 (SEQ ID NO:12), CYP76AH11 (SEQ ID NO:9) and CYP76AH16 (SEQ ID NO:2) also produces a compound co-eluating with deacetylforskolin.

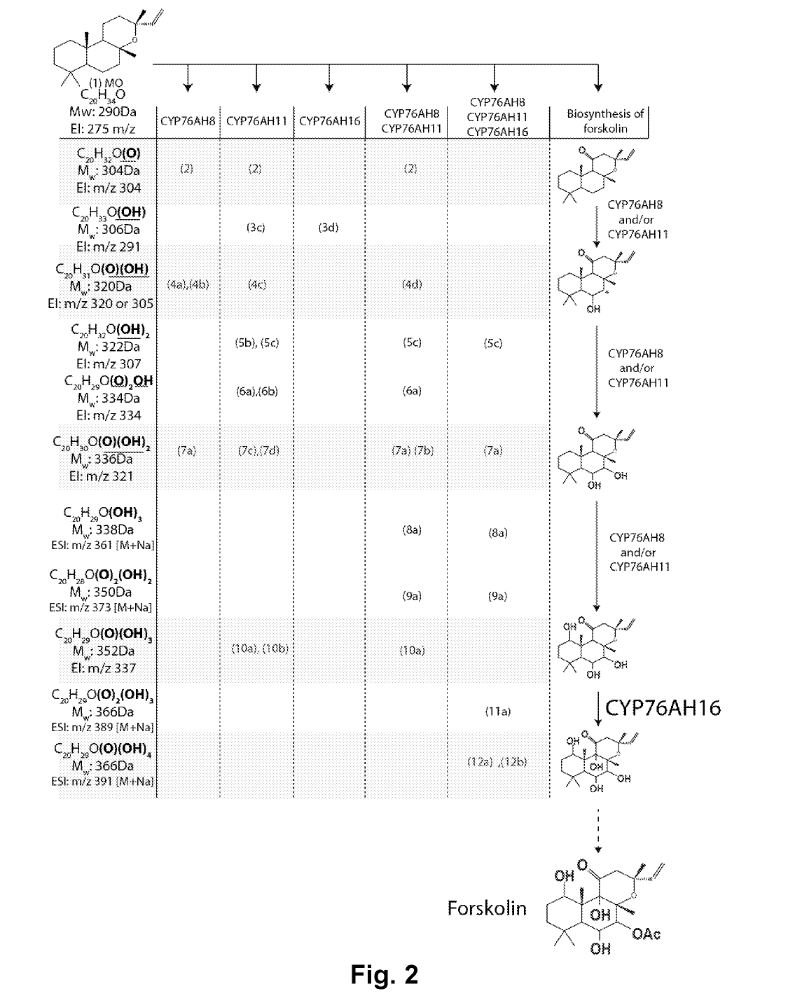

A summary of the oxidation reactions of 13R-MO en route to forskolin are shown in FIG. 2. CYP76AH8 and CYP76AH11 effectively catalyse the first three reactions, whereas CYP76AH16 effectively catalyse hydroxylation of the 9 position of 13R-MO. Similar results are observed if either CYP76AH15 or CYP76AH17 are expressed in stead of CYP76AH8.

### Example 2

All chemicals were acquired from Sigma-Aldrich. Preparation of the authentic standard of 13R-(+)-manoyl oxide was described Thrane et al 2014.

http://dxdoi.org/10.1021/sb500055u

For functional characterization of CYPs from Coleus forskohlii (CfCYPS) and testing of their ability to oxygenate manoyl oxide, CfCYPs was transient expressed in N. benthamiana with and without co-expression of Coleus forskohlii 1-deoxy-d-xylulose 5-phosphate synthase (CfDXS—SEQ ID NO:7), geranylgeranyl diphosphate synthase CfGGPPs (SEQ ID NO:6), CfTPS2 (SEQ ID NO:3), CfTPS3 (SEQ ID NO:4) involved in high level biosynthesis of 13R-(+)-manoyl oxide. Transient expression in N. benthamiana was done using the protocol described in Spanner et al. 2014 PMID: 24777803). CfCYP genes selected for functional testing in N. benthamiana was isolated from Coleus forskohlii cDNA and introduced into pCAMBIA130035Su by USER cloning as described in Nour et al. http://dx.doi.org/10.1007/978-1-60761-723-5 13) and transformed into agrobacteria strain AGL-1-GC3850 as described in Spanner et al 2014 PMID: 24777803. For infiltration, the OD600 of all transformed agrobacteria strains was normalized to OD600=1. Agrobacteria strains containing the pCAMBIA130035Su with genes involved in high level biosynthesis of 1 was mixed. Each of the CfCYPs selected for functional testing was subsequently added to the agrobacteria mixture in independent mixtures and infiltrated into 4-6 weeks old N. benthamiana plant.

The CYPs expressed were the following:

CYP76AH16 of SEQ ID NO:2

CYP76AH8 of SEQ ID NO:8

CYP76AH11 of SEQ ID NO:9

CYP76AH15 of SEQ ID NO:13

CYP76AH17 of SEQ ID NO:12

Extracts of the N. benthamiana transiently expressing the indicated enzymes were analysed by LC-MS-qTOF and/or GS-MS.

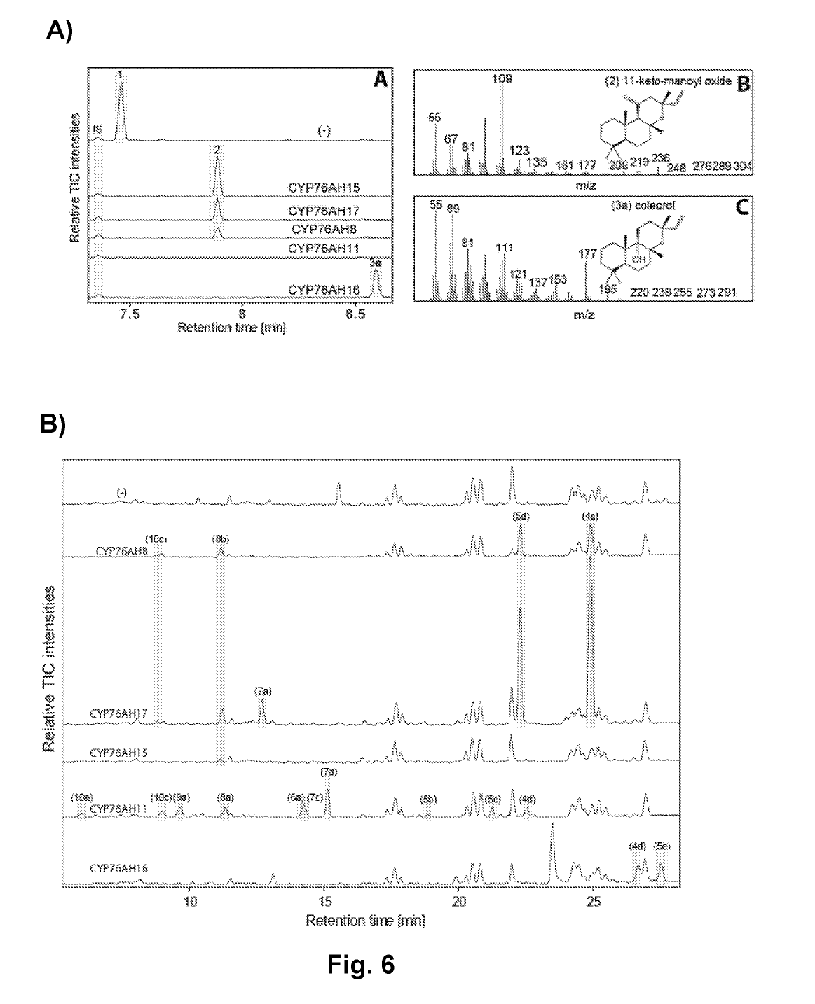

The results of GC-MS analysis of extracts from N. benthamiana transiently expressing CfCXS, CfGGPPs, CfTPS2 and CfTPS3 in combination with water (−), CYP76AH15, CYP76AH17, CYP76AH8, CYP76AH11 or CYP76AH16, respectively are shown in FIG. 6

An overview of the compounds produced by expressing single CYPs and different combinations of the CYPs is shown in FIGS. 8 and 7, respectively.

The results show that the ketone formation at C-11 can be catalyzed alternatively by 3 different CYPs which belong to the subfamily CYP76AH, the CYP76AH15 of SEQ ID NO:13, CYP76AH8 of SEQ ID NO:8 and CYP76AH17 of SEQ ID NO:12. Although all 3 above mentioned CYPs are able to catalyze independently the 11-ketonation of MO, each one exhibits different efficiency/specificity towards this reaction, with the CYP76AH15 showing the highest specificity towards forskolin precursor 11-keto-MO, as the detected peak for 11-keto-MO is higher for this enzyme in comparison to the others and at the same time less side products are observed (see FIG. 8). From these compounds, the structures of the 5d and 10c have been elucidated using NMR (FIG. 8), and it was shown that in both of them the positions of hydroxylations occurred by the specific CYPs coincide with hydroxylation in the forskolin structure. Interestingly compound 10c produced by expression of CYP76AH8 of SEQ ID NO:8 or CYP76AH17 of SEQ ID NO:12, although a minor peak, includes the 11-keto group together with 3 out of 4 hydroxyl groups observed in forskolin, at positions C-1, C-6 and C-7.

Two enzymes showed catalytic activity related to forskolin biosynthesis. These enzymes, CYP76AH11 of SEQ ID NO:9 and CYP76AH16 of SEQ ID NO:2, come from the same subfamily CYP76AH. CYP76AH16 of SEQ ID NO:2 proved to be a quite specific enzyme and hydroxylates almost exclusively the C-9 (FIG. 6 and FIG. 8). The enzyme CYP76AH11 of SEQ ID NO:9 exhibited a multiple function, as it is possible to catalyze multiple hydroxylations at C-1, C-6 and C-7 (FIG. 6 and FIG. 8). The experiments presented here shows that co-expression of CYP76AH11 of SEQ ID NO:9 and CYP76AH16 of SEQ ID NO:2 together with the CYP76AH8 SEQ D NO:8 or CYP76AH17 of SEQ ID NO:12 or CYP76AH15 of SEQ ID NO:13 as shown in FIG. 7 can result to the synthesis of deacetyl-forskolin.

1. A method of producing an oxidised 13R-manoyl oxide (13R-MO), the method comprising:
(a) providing a host organism. comprising a heterologous nucleic acid encoding an enzyme capable of catalysing hydroxylation of 13R-MO and/or oxidised 13R-MO at the 9 position, wherein the oxidised 13R-MO carries a —H at the 9-position;
(b) incubating the host organism in the presence of 13R-MO under conditions allowing growth of the host organism; and
(c) optionally isolating the oxidised 13R-MO from the host organism.

2. The method of claim 1, wherein the oxidised 13-R-MO is:
(a) a compound of formula: 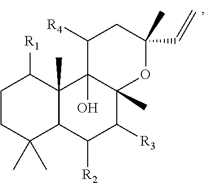
wherein R₁, R₂, and R₃ individually are selected from the group consisting of —H, —OH and —OR, R is acyl, and R₄ is selected from the group consisting of —H, —OH and ═O;
(b) a compound of the formula: 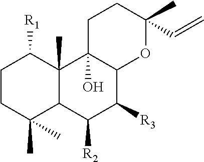
wherein R₁, R2, and R₃ individually are selected from the group consisting of —H, —OH and —OR and R is acyl;
(c) a compound of the formula: 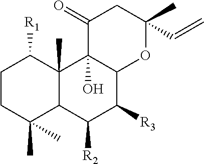
wherein R₁, R2, and R₃ individually are selected from the group consisting of —H, —OH and —OR and R is acyl.

3-4. (canceled)

5. The method of claim 2, wherein:
(a) R₁ is selected from the group consisting of —H and —OH;
(b) R2 is selected from the group consisting of —OR and —OH and R is acyl;
(c) R3 is selected from the group consisting of —OR and —OH, and R is acyl; and
(d) R4 is selected from the group consisting of —H, ═O and —OH.

6-8. (canceled)

9. The method of claim 1, wherein the oxidised 13R-MO is deacetyl-forskolin and/or forskolin.

10. (canceled)

11. A host organism comprising a heterologous nucleic acid encoding an enzyme capable of catalysing hydroxylation of 13R-MO and/or oxidised 13R-MO at the 9 position, wherein the oxidised 13R-MO carries a —H at the 9-position.

12. The host organism of claim 11, wherein the host organism further comprises:
(a) a heterologous nucleic acid encoding an enzyme capable of catalysing hydroxylation of 13R-manoyl oxide (13R-MO) and/or an oxidised 13R-MO derivative at the 11 position, wherein the oxidised 13R-MO carries a —H at the 11-position; and/or catalysing oxidation of the hydroxyl group to form an oxo-group at the 11 position of 11-hydroxyl-13R-MO and/or an oxidised 11-hydroxyl-13R-MO;
(b) a heterologous nucleic acid encoding an enzyme capable of catalysing hydroxylation of 13R-MO and/or oxidised 13R-MO at the 1 position, wherein the oxidised 13R-MO carries a —H at the 1-position;
(c) a heterologous nucleic acid encoding an enzyme capable of catalysing hydroxylation of 13R-MO and/or oxidised 13R-MO at the 6 position, wherein the oxidised 13R-MO carries a —H at the 6-position;
(d) a heterologous nucleic acid encoding an enzyme capable of catalysing hydroxylation of 13R-MO and/or oxidised 13R-MO at the 7 position, wherein the oxidised 13R-MO carries a —H at the 7-position;
(e) a heterologous nucleic acid encoding an enzyme capable of catalysing transfer of an acyl group to an —OH of a hydroxylated 13R-MO and/or an oxidised hydroxylated-13R-MO;
(f) a heterologous nucleic acid encoding TPS2;
(g) a heterologous nucleic acid encoding TPS3; and/or
(h) a heterologous nucleic acid encoding TPS4.

13-14. (canceled)

15. The host organism of claim 11, further comprising:
(a) a heterologous nucleic acid encoding an enzyme involved in the synthesis of GGPP; and/or
(b) a heteroloqous nucleic acid encoding a 1-deoxy-D-xylulose-5-phosphate synthase.

16. The host organism of claim 15, wherein the enzyme involved in the synthesis of GGPP is a GGPP synthase having at least 70% sequence identity to the amino acid sequence set forth in SEQ ID NO:6.

17-18. (canceled)

19. The host organism of claim 15, wherein the 1-deoxy-D-xylulose-5-phosphate synthase has at least 70% sequence identity to the amino acid sequence set forth in SEQ ID NO:7.

20. (canceled)

21. A polypeptide having at least 70% sequence identity to the amino acid sequence set forth in SEQ ID NO:2, wherein the polypeptide is an enzyme capable of catalysing hydroxylation of 13R-MO and/or oxidised 13R-MO at the 9 position, wherein the oxidised 13R-MO carries a —H at the 9-position at the 9-position.

22-24. (canceled)

25. The method of claim 1, wherein the enzyme capable of catalysing hydroxylation of 13R-MO and/or oxidised 13R-MO at the 9 position has at least 70% sequence identity to the amino acid sequence set forth in SEQ ID NO:11.

26. The host organism of claim 11, wherein the enzyme capable of catalysing hydroxylation of 13R-MO and/or oxidised 13R-MO at the 9 position has at least 70% sequence identity to the amino acid sequence set forth in SEQ ID NO:11.

27. (canceled)

28. The method of claim 1, wherein the host organism further comprises:
(a) a heteroloqous nucleic acid encoding and enzyme having at least 70% sequence identity to the amino acid sequence set forth in SEQ ID NO:8;
(b) a heteroloqous nucleic acid encoding and enzyme having at least 70% sequence identity to the amino acid sequence set forth in SEQ ID NO:9;
(c) a heteroloqous nucleic acid encoding and enzyme having at least 70% sequence identity to the amino acid sequence set forth in SEQ ID NO:11;
(d) a heteroloqous nucleic acid encoding and enzyme having at least 70% sequence identity to the amino acid sequence set forth in SEQ ID NO:12; and/or
(e) a heteroloqous nucleic acid encoding and enzyme having at least 70% sequence identity to the amino acid sequence set forth in SEQ ID NO: 13.

29. The host organism of claim 11, wherein the host organism further comprises:
(a) a heterologous nucleic acid encoding and enzyme having at least 70% sequence identity to the amino acid sequence set forth in SEQ ID NO:8;
(b) a heterologous nucleic acid encoding and enzyme having at least 70% sequence identity to the amino acid sequence set forth in SEQ ID NO:9;
(c) a heterologous nucleic acid encoding and enzyme having at least 70% sequence identity to the amino acid sequence set forth in SEQ ID NO:11;
(d) a heterologous nucleic acid encoding and enzyme having at least 70% sequence identity to the amino acid sequence set forth in SEQ ID NO:12; and/or
(e) a heterologous nucleic acid encoding and enzyme having at least 70% sequence identity to the amino acid sequence set forth in SEQ ID NO: 13.

30-35. (canceled)

36. The host organism of claim 11, wherein:
(a) the TPSP2 has at least 70% sequence identity to the amino acid sequence set forth in SEQ ID NO:3;
(b) the TPSP3 has at least 70% sequence identity to the amino acid sequence set forth in SEQ ID NO:4; and
(c) the TPSP3 has at least 70% sequence identity to the amino acid sequence set forth in SEQ ID NO:5.

37-38. (canceled)

39. The host organism of claim 11, wherein the host organism is a microorganism.

40. The method or the host organism of claim 39, wherein the microorganism is yeast.

41. The host organism of claim 11, wherein the host organism is a plant.

42. A method of producing an oxidised 13R-MO, the method comprising:
(a) providing the host organism of claim 11;
(b) preparing an extract of the host organism;
(c) providing 13R-MO; and
(d) incubating the extract with 13R-MO; thereby producing the oxidised 13R-MO.
